(19) United States
(12) Patent Application Publication
(10) Pub. No.: US 2026/0334999 A1
(43) Pub. Date: Oct. 29, 2026

(54) Electrosurgical Systems, Devices, and Methods with Features for Enhanced Visualization and/or Maneuverability

(71) Applicant: Stryker European Operations Limited, Cork (IE)

(72) Inventors: Rahul Padman, Cork (IE); Syed Ashad Mustufa Younus, Cork (IE); Mark Sheridan, Ardnacrusha (IE); Micheal Burke, Cork (IE); Paul Sheridan, Wexford (IE); Brandon Laure, Satellite Beach, FL (US); Brian Vanderwoude, Portage, MI (US)

(21) Appl. No.: 19/486,253

(22) PCT Filed: Dec. 18, 2024
(86) PCT No.: PCT/IB2024/000722

§ 371 Date: Nov. 20, 2025

Related U.S. Application Data
(60) Provisional application No. 63/611,492, filed on Dec. 18, 2023.

(51) Int. Cl. A61B 18/14; A61B 17/00; A61B 18/00; A61B 18/12; A61B 90/00; A61B 90/30

(52) U.S. Cl. CPC A61B 18/14; A61B 18/1206; A61B 90/30; A61B 90/37; A61B 2017/00221; A61B 2017/00526; A61B 2018/00077; A61B 2018/00083; A61B 2018/00178; A61B 2018/00184; A61B 2018/00589; A61B 2018/00601; A61B 2018/00607; A61B 2018/00702; A61B 2018/00916; A61B 2018/00982

(57) ABSTRACT

An example electrosurgical device includes a handle extending between a proximal handle end and a distal handle end. The electrosurgical device also includes a shaft extending from the distal handle end of the handle. The shaft extends between a proximal shaft end and a distal shaft end. The electrosurgical device further includes a camera at the distal shaft end, and an electrosurgical electrode coupled to an exterior surface the shaft and extending distally of the distal shaft end. A longitudinal axis of the electrosurgical electrode is parallel to a longitudinal axis of the shaft.

### CROSS-REFERENCE TO RELATED APPLICATIONS

The present application claims the benefit of priority of U.S. Provisional Application No. 63/611,492, filed Dec. 18, 2023, the contents of which are hereby incorporated by reference in their entirety.

### BACKGROUND

Electrosurgery involves applying a radio frequency (RF) electric current (also referred to as electrosurgical energy) to biological tissue to cut, coagulate, or modify the biological tissue during an electrosurgical procedure. Specifically, an electrosurgical generator generates and provides the electric current to an active electrode, which applies the electric current (and, thus, electrical power) to the tissue. The electric current passes through the tissue and returns to the generator via a return electrode (also referred to as a “dispersive electrode”). As the electric current passes through the tissue, an impedance of the tissue converts a portion of the electric current into thermal energy (e.g., via the principles of resistive heating), which increases a temperature of the tissue and induces modifications to the tissue (e.g., cutting, coagulating, ablating, and/or sealing the tissue)..

### BRIEF DESCRIPTION OF THE FIGURES

The novel features believed characteristic of the illustrative embodiments are set forth in the appended claims. The illustrative embodiments, however, as well as a preferred mode of use, further objectives and descriptions thereof, will best be understood by reference to the following detailed description of an illustrative implementation of the present disclosure when read in conjunction with the accompanying figures, wherein:

FIG. 1 depicts a simplified block diagram of an electrosurgical system, according to an example.

FIG. 2A depicts a perspective view of an electrosurgical device, according to an example.

FIG. 2B depicts an enlarged view of a distal portion of the electrosurgical device shown in FIG. 2A, according to an example.

FIG. 2C depicts a partial cross-sectional view of the electrosurgical device shown in FIG. 2A, according to an example.

FIG. 2D depicts a perspective view of a camera assembly shown in FIGS. 2A-2C, according to an example.

FIG. 3A depicts a perspective view of an electrosurgical device, according to another example.

FIG. 3B depicts an enlarged view of a distal portion of the electrosurgical device shown in FIG. 3A, according to an example.

FIG. 3C depicts a partial cross-sectional view of a collar shown in FIGS. 3A-3B, according to an example.

FIG. 4A depicts a camera assembly, according to an example.

FIG. 4B depicts the camera assembly of FIG. 4A coupled to a shaft of an electrosurgical device, according to an example.

FIG. 5 depicts a camera assembly coupled to handle of an electrosurgical device, according to an example.

FIG. 6A depicts an electrosurgical device and a camera assembly, according to an example.

FIG. 6B depicts a portion of a handle of the electrosurgical device shown in FIG. 6A, according to an example.

FIG. 6C depicts the camera assembly of FIG. 6A in a first state, according to an example.

FIG. 6D depicts the camera assembly of FIG. 6B in a second state, according to an example.

FIG. 7A depicts an electrosurgical device, according to another example.

FIG. 7B depicts a cross-sectional view of a handle of the electrosurgical device shown in FIG. 7A, according to an example.

FIG. 8A depicts an electrosurgical device, according to another example.

FIG. 8B depicts a cross-sectional view of a handle of the electrosurgical device shown in FIG. 8A, according to an example.

FIG. 8C depicts a cross-sectional view of the handle taken through a line in shown in FIG. 8B and with an exterior housing portion in the locked position, according to an example.

FIG. 8D depicts a cross-sectional view of the handle through a line in shown in FIG. 8B and with the exterior housing portion in the unlocked position, according to an example.

FIG. 9A depicts an electrosurgical device, according to another example.

FIG. 9B depicts a cross-sectional view of a handle of the electrosurgical device shown in FIG. 9A, according to an example.

FIG. 9C depicts a cross-sectional view of the handle taken through a line in shown in FIG. 9B, according to an example.

FIG. 10 depicts a camera assembly, according to another exmaple.

FIG. 11A depicts a simplified block diagram of one or more cameras communicatively coupled to a controller, according to an example.

FIG. 11B depicts a front end view of an electrosurgical device including a plurality of cameras, according to an example.

FIG. 12 depicts a simplified block diagram of an electrosurgical system, according to an example.

FIG. 13 depicts a simplified block diagram of an electrosurgical system, according to an example.

FIG. 14A depicts a side view of an electrosurgical device, according to another example.

FIG. 14B depicts a simplified circuit schematic diagram of the electrosurgical device shown in FIG. 14A, according to the example.

FIG. 15 depicts a flowchart for a process of operating an electrosurgical device, according to an example..

FIG. 16 depicts a flowchart for a process of operating an electrosurgical device that can be used with the process shown in at least FIG. 15, according to an example.

FIG. 17 depicts a flowchart for a process of operating an electrosurgical device that can be used with the process shown in at least FIG. 16, according to an example.

FIG. 18 depicts a flowchart for a process of operating an electrosurgical device that can be used with the process shown in at least FIG. 15, according to an example.

FIG. 19 depicts a flowchart for a process of operating an electrosurgical device that can be used with the process shown in at least FIG. 15, according to an example.

FIG. 20 depicts a flowchart for a process of operating an electrosurgical device that can be used with the process shown in at least FIG. 15, according to an example.

FIG. 21 depicts a flowchart for a process of operating an electrosurgical device that can be used with the process shown in at least FIG. 15, according to an example.

FIG. 22 depicts a flowchart for a process of operating an electrosurgical device that can be used with the process shown in at least FIG. 15, according to an example.

FIG. 23 depicts a flowchart for a process of operating an electrosurgical device that can be used with the process shown in at least FIG. 15, according to an example.

FIG. 24 depicts a flowchart for a process of operating an electrosurgical device that can be used with the process shown in at least FIG. 23, according to an example.

FIG. 25 depicts a flowchart for a process of operating an electrosurgical device, according to another example.

FIG. 26 depicts a flowchart for a process of operating an electrosurgical device that can be used with the process shown in at least FIG. 25, according to an example.

FIG. 27 depicts a flowchart for a process of operating an electrosurgical device that can be used with the process shown in at least FIG. 26, according to an example.

FIG. 28 depicts a flowchart for a process of operating an electrosurgical device that can be used with the process shown in at least FIG. 25, according to an example.

FIG. 29 depicts a flowchart for a process of operating an electrosurgical device that can be used with the process shown in at least FIG. 25, according to an example.

FIG. 30 depicts a flowchart for a process of operating an electrosurgical device that can be used with the process shown in at least FIG. 25, according to an example.

FIG. 31 depicts a flowchart for a process of operating an electrosurgical device that can be used with the process shown in at least FIG. 25, according to an example.

FIG. 32 depicts a flowchart for a process of operating an electrosurgical device that can be used with the process shown in at least FIG. 25, according to an example.

FIG. 33 depicts a flowchart for a process of operating an electrosurgical device that can be used with the process shown in at least FIG. 25, according to an example.

FIG. 34 depicts a flowchart for a process of operating an electrosurgical device that can be used with the process shown in at least FIG. 33, according to an example.

FIG. 35 depicts a flowchart for a process of forming an electrosurgical device, according to another example.

FIG. 36 depicts a flowchart for a process of forming an electrosurgical device, according to another example.

### DESCRIPTION

Disclosed examples will now be described more fully hereinafter with reference to the accompanying drawings, in which some, but not all of the disclosed examples are shown. Indeed, several different examples may be described and should not be construed as limited to the examples set forth herein. Rather, these examples are described so that this disclosure will be thorough and complete and will fully convey the scope of the disclosure to those skilled in the art.

By the term “approximately” or “substantially” with reference to amounts or measurement values described herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

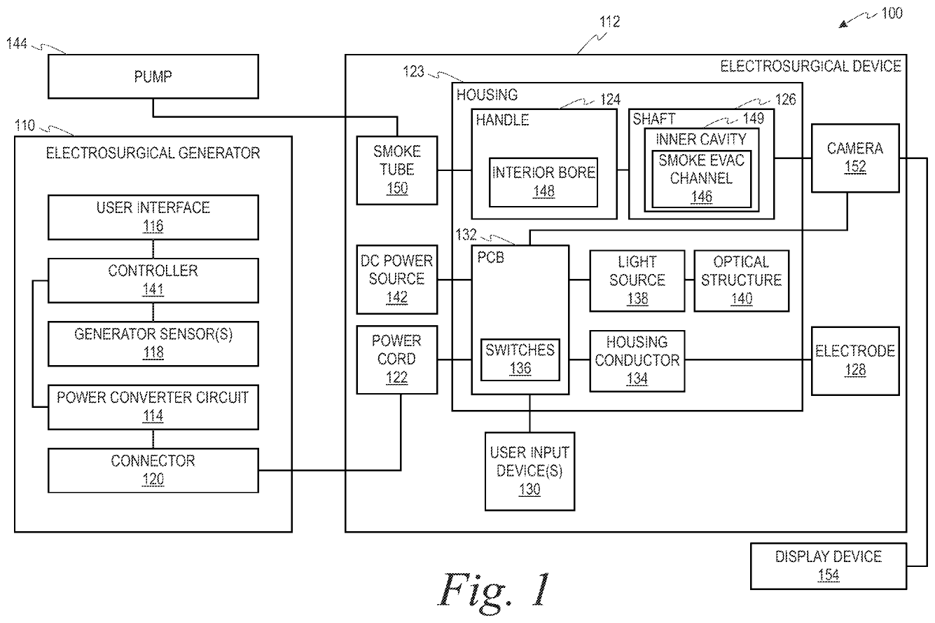

Referring to FIG. 1, an electrosurgical system 100 is shown according to an example. As shown in FIG. 1, the electrosurgical system 100 includes an electrosurgical generator 110 and an electrosurgical device 112. In general, the electrosurgical generator 110 can generate electrosurgical energy that is suitable for performing electrosurgery on a patient. For instance, the electrosurgical generator 110 can include a power converter circuit 114 that can convert a grid power to electrosurgical energy such as, for example, a radio frequency (RF) output power. As an example, the power converter circuit 114 can include one or more electrical components (e.g., one or more transformers) that can control a voltage, a current, and/or a frequency of the electrosurgical energy.

Within examples, the electrosurgical generator 110 can include a user interface 116 that can receive one or more inputs from a user and/or provide one or more outputs to the user. As examples, the user interface 116 can include one or more buttons, one or more switches, one or more dials, one or more keypads, one or more touchscreens, one or more display screens, one or more indicator lights, one or more speakers, and/or one or more haptic output devices.

In an example, the user interface 116 can be operable to select a mode of operation from among a plurality of modes of operation for the electrosurgical generator 110. As examples, the modes of operation can include a cutting mode, a coagulating mode, an ablating mode, and/or a sealing mode. Combinations of these waveforms can also be formed to create blended modes. In one implementation, the modes of operation can correspond to respective waveforms for the electrosurgical energy. As such, in this implementation, the electrosurgical generator 110 can generate the electrosurgical energy with a waveform selected from a plurality of waveforms based, at least in part, on the mode of operation selected using the user interface 116.

The electrosurgical generator 110 can also include one or more generator sensors 118 that can sense one or more conditions related to the electrosurgical energy and/or the target tissue. As examples, the generator sensor(s) 118 can include one or more current sensors, one or more voltage sensors, one or more temperature sensors, and/or one or more bioimpedance sensors. Within examples, the electrosurgical generator 110 can additionally or alternatively generate the electrosurgical energy with an amount of electrosurgical energy (e.g., an electrical power) and/or a waveform selected from among the plurality of waveforms based on one or more parameters related to the condition(s) sensed by the generator sensor(s) 118.

In one example, the electrosurgical energy can have a frequency that is greater than approximately 100 kilohertz (kHz) to reduce (or avoid) stimulating a muscle and/or a nerve near the target tissue. In another example, the electrosurgical energy can have a frequency that is between approximately 300 kHz and approximately 500 kHz.

In FIG. 1, the electrosurgical generator 110 also includes a connector 120 that can facilitate coupling the electrosurgical generator 110 to the electrosurgical device 112. For example, the electrosurgical device 112 can include a power cord 122 having a plug, which can be coupled to a socket of the connector 120 of the electrosurgical generator 110. In this arrangement, the electrosurgical generator 110 can supply the electrosurgical energy to the electrosurgical device 112 via the coupling between the connector 120 of the electrosurgical generator 110 and the power cord 122 of the electrosurgical device 112.

The electrosurgical generator 110 can further include a controller 141 that can control operation of the electrosurgical generator 110. Within examples, the controller 141 can be implemented using hardware, software, and/or firmware. For instance, the controller 141 can include one or more processors and a non-transitory computer readable medium (e.g., volatile and/or non-volatile memory) that stores machine language instructions or other executable instructions. The instructions, when executed by the one or more processors, cause the electrosurgical generator 110 to carry out the various operations described herein. The controller 141, thus, can receive data and store the data in the memory as well. As shown in FIG. 1, the controller 141 can be communicatively coupled with the power converter circuit 114, the user interface 116, the generator sensor(s) 118, and/or the connector 120.

As shown in FIG. 1, the electrosurgical device 112 can include a housing 123 having a proximal end and a distal end, and an electrosurgical electrode 128 extending from the distal end of the housing 123. The housing 123 can be an elongated structure in and/or on which components of the electrosurgical device 112 can be disposed. In some examples, the housing 123 can be an integral, monolithic structure. In other examples the housing 123 can include a plurality of structures that are coupled to each other.

In FIG. 1, the housing 123 includes a handle 124 that defines an interior bore, and a shaft 126 extending in a distal direction from the handle 124. In general, the handle 124 can be configured to facilitate a user gripping and manipulating the electrosurgical device 112 while performing electrosurgery. For example, the handle 124 can have a shape and/or a size that can facilitate a user performing electrosurgery by manipulating the electrosurgical device 112 using a single hand. In one implementation, the handle 124 can have a shape and/or a size that facilitates the user holding the electrosurgical device 112 in a writing utensil gripping manner (e.g., the electrosurgical device 112 can be an electrosurgical pencil).

Additionally, for example, the handle 124 and/or the shaft 126 can be constructed from one or more materials that are electrical insulators (e.g., a plastic material). This can facilitate insulating the user from the electrosurgical energy flowing through the electrosurgical device 112 while performing the electrosurgery.

In some implementations, the shaft 126 can be coupled to the handle 124 in a fixed and non-moveable manner. This may simplify manufacturing and reduce a cost of manufacture by, for instance, simplifying electrical connections that may otherwise need to account for movement of the shaft 126 and the handle 124 relative to each other (e.g., by omitting slip ring electrical contacts and/or sliding electrical contacts). In one example, the handle 124 and the shaft 126 can be formed as a single, monolithic structure such that the shaft 126 and the handle 124 are fixed and non-moveable relative to each other. In another example, the handle 124 and the shaft 126 can be fixedly coupled to each other by a welding coupling, an adhesive coupling, and/or another coupling that prevents movement between the handle 124 and the shaft 126.

In other implementations, the shaft 126 can be telescopically moveable relative to the handle 124. For example, the shaft 126 can be telescopically moveable in the interior bore defined by the handle 124 to extend the shaft 126 in the distal direction and retract the shaft 126 in a proximal direction relative to the handle 124 (e.g., movable along a longitudinal axis of the electrosurgical device 112). In some examples, the electrosurgical electrode 128 can be coupled to the shaft 126 and, thus, the electrosurgical electrode 128 can move together with the shaft 126 in an axial direction along the longitudinal axis relative to the handle 124. This can provide for adjusting a length of the electrosurgical device 112, which can facilitate performing electrosurgery at a plurality of different depths within tissue (e.g., due to different anatomical shapes and/or sizes of patients) and/or at a plurality of different angles. In other examples, the electrosurgical electrode 128 can be fixedly coupled to the handle 124 such that the shaft 126 is axially movable relative to both the electrosurgical electrode 128 and the handle 124.

In some implementations, the electrosurgical electrode 128 can additionally or alternatively be rotatable about an axis of rotation that is parallel to the longitudinal axis of the electrosurgical device 112. In some examples, the electrosurgical electrode 128 can be rotatable relative to the handle 124 and the shaft 126. In other examples, the electrosurgical electrode 128 can be rotationally fixed relative to the shaft 126 such that the shaft 126 and the electrosurgical electrode 128 are rotatable together relative to the handle 124. Rotating the electrosurgical electrode 128 relative to the handle 124 can facilitate adjusting an angle of the electrosurgical electrode 128 relative to one or more user input device(s) 130 of the electrosurgical device 112. In this arrangement, a user can comfortably grip the handle 124 in a position in which their fingers can comfortably operate the user input device(s) 130 while the electrosurgical electrode 128 is set at a rotational position selected from among a plurality of rotational positions relative to the handle 124 based on, for example, a location, a size, and/or a shape of a surgical site in which the user is operating.

In one implementation, the electrosurgical electrode 128 can be rotatable by more than 360 degrees relative to the handle 124. This can improve an ease of use by allowing an operator to freely rotate the electrosurgical electrode 128 without limitation. However, in other implementations, the electrosurgical electrode 128 can be rotatable by less than or equal to 360 degrees (e.g., rotatable by 180 degrees, rotatable by 270 degrees, or rotatable by 360 degrees). This may still allow an operator to achieve a desired rotational arrangement, but with the possibility that the operator may rotate in first direction, reach a stop limiting further rotation, and then rotate back in a second direction to achieve the desired rotational arrangement.

Although it can be beneficial to provide for rotation of the electrosurgical electrode 128 relative to the handle 124 and/or the shaft 126, the electrosurgical electrode 128 can be rotationally fixed relative to the handle 124 and the shaft 126 in some implementations. This may, for example, help to simplify manufacturing and reduce a cost of manufacture by, for instance, simplifying electrical connections that may otherwise need to account for movement of the shaft 126 and the handle 124 relative to each other (e.g., by omitting slip ring electrical contacts and/or sliding electrical contacts).

As shown in FIG. 1, the electrosurgical device 112 can include one or more user input devices 130 that are operable to control operation of the electrosurgical device 112 and/or the electrosurgical generator 110. For instance, the user input device(s) 130 can be operable to select between the modes of operation of the electrosurgical device 112 and/or the electrosurgical generator 110. In one implementation, the user input device(s) 130 can be configured to select between a cutting mode of operation and a coagulation mode of operation. Responsive to actuation of the user input device(s) 130 of the electrosurgical device 112, the electrosurgical device 112 can (i) receive the electrosurgical energy with a level of power and/or a waveform corresponding to the mode of operation selected via the user input device(s) 130 and (ii) supply the electrosurgical energy to the electrosurgical electrode 128.

In FIG. 1, the electrosurgical device 112 includes a plurality of electrical components that facilitate supplying the electrosurgical energy, which the electrosurgical device 112 receives from the electrosurgical generator 110, to the electrosurgical electrode 128. For example, the electrosurgical device 112 can include at least one electrical component selected from a group of electrical components including: a printed circuit board 132 (e.g., a flexible printed circuit board) and/or one or more housing conductors 134 that are configured to conduct electrosurgical energy from the power cord 122 to the electrosurgical electrode 128. One or more of the electrical components can be positioned in the interior bore 148 defined by the handle 124 and/or in an inner cavity 149 defined by the shaft 126.

Within examples, the user input device(s) 130 can include one or more buttons on an exterior surface of the handle 124. Each button of the user input device(s) 130 can be operable to actuate a respective one of a plurality of switches 136 of the printed circuit board 132. In general, the switches 136 and/or the printed circuit board 132 are operable to control a supply of the electrosurgical energy from the electrosurgical generator 110 to the electrosurgical electrode 128. For instance, in one implementation, when each button is operated (e.g., depressed), the respective switch 136 associated with the button can be actuated to cause the printed circuit board 132 to transmit a signal to the electrosurgical generator 110 and cause the electrosurgical generator 110 to responsively supply the electrosurgical energy with a level of power and/or a waveform corresponding to a mode of operation associated with the button. In another implementation, operating the button and thereby actuating the respective switch 136 associated with the button can close the switch 136 to complete a circuit to the electrosurgical generator 110 to cause the electrosurgical generator 110 to responsively supply the electrosurgical energy with a level of power and/or a waveform corresponding to a mode of operation associated with the button. In some examples of this implementation, the printed circuit board 132 can be omitted.

In both example implementations, the electrosurgical energy supplied by the electrosurgical generator 110 can be supplied from (i) the power cord 122, the printed circuit board 132, and/or the switch(es) 136 to (ii) the electrosurgical electrode 128 by the housing conductor(s) 134. As such, as shown in FIG. 1, the printed circuit board 132 can be coupled to the power cord 122, the printed circuit board 132 can be coupled to the housing conductor(s) 134, and the housing conductor(s) 134 can be coupled to the electrosurgical electrode 128. In this arrangement, the housing conductor(s) 134 can conduct the electrosurgical energy to the electrosurgical electrode 128. The switch(es) 136 can be coupled to the printed circuit board 132 in some examples.

In general, the housing conductor(s) 134 can each include one or more electrically conductive elements that provide an electrically conductive bus for supplying the electrosurgical energy to the electrosurgical electrode 128. In some examples, the electrical components of the electrosurgical device 112 can be electrically coupled to each other in a manner that is suitable to supply electrosurgical energy from the power cord 122 to the electrosurgical electrode 128 while (i) the shaft 126 and/or the electrosurgical electrode 128 telescopically moves relative to the handle 124, and/or (ii) the electrosurgical electrode 128 rotates relative to the handle 124.

Although the electrosurgical device 112 includes the user input device(s) 130 in FIG. 1, the user input device(s) 130 can be separate from the electrosurgical device 112 in another example. For instance, the user input device(s) 130 can additionally or alternatively include one or more foot pedals that are actuatable to control operation of the electrosurgical device 112 as described above. The foot pedal(s) can be communicatively coupled to the electrosurgical generator 110 to provide a signal responsive to actuation of the foot pedal(s).

As shown in FIG. 1, in some implementations, the electrosurgical device 112 can additionally include one or more light sources 138 that are configured to emit light. In some examples that include the light source(s) 138, the user input device 130 can be operable to cause the light source(s) 138 to generate light that can be emitted by the electrosurgical device 112 to illuminate an area of interest (e.g., a target tissue at the surgical site). In some implementations, the light source(s) 138 can be located at a distal end of the housing 123 and/or a distal end of the shaft 126 to directly provide light in a distal direction and illuminate a surgical distal of the electrosurgical electrode 128.

In other implementations, as shown in FIG. 1, the light source(s) 138 can be optically coupled to an optical structure 140, which is configured to receive the light emitted by the light source(s) 138 and transmit the light in a distal direction toward a surgical site to illuminate the surgical site while performing electrosurgery using the electrosurgical electrode 128. Although arranging the light source(s) 138 to directly illuminate a surgical field can help, for instance, to reduce a cost of manufacture, transmitting the light using the optical structure 140 can help to improve a quality of light transmitted from the electrosurgical device 112 (e.g., by providing light with improved uniformity and/or reduced heat generation).

As examples, in implementations that include the optical structure 140, the optical structure 140 can include at least one optical structure selected from among a group consisting of an optical lens, a non-fiber optic optical waveguide, and an optical fiber. When the optical structure 140 includes the optical lens (e.g., a parabolic reflector lens, an aspheric lens, and/or a Fresnel lens), the optical structure 140 can help to direct the light emitted by the light source 138 in the distal direction and thereby improve a quality of the light illuminating the surgical site. The optical structure 140 can additionally or alternatively include the non-fiber optic optical waveguide and/or the optical fiber to transmit the light over relatively large distances in the shaft 126. For instance, the optical waveguide can transmit the light in the distal direction via total internal reflection. In such implementations, the optical waveguide can include a cladding and/or an air gap on an exterior surface of the optical waveguide to help facilitate total internal reflection. In some implementations, the non-fiber optic optical waveguide can be formed as a single, monolithic structure.

In some examples, the optical structure 140 can additionally or alternatively include other light shaping optical elements such as, for instance, a plurality of facets, one or more prisms, and/or one or more optical gratings. Although the optical structure 140 can help to improve a quality of the light directed to the surgical site, the electrosurgical device 112 can omit the optical structure 140 and instead emit the light from the light source 138 directly to the surgical field without transmitting the light through the optical structure 140 in other examples.

In FIG. 1, the light source 138 can be coupled to the shaft 126. As such, the light source 138 can also move telescopically with the shaft 126 relative to the handle 124. However, in other examples, the light source 138 can be in the interior bore of the handle 124 and/or coupled to an exterior surface of the handle 124. As examples, the light source 138 can include one or more light emitting diodes (LEDs), organic light emitting diodes (OLEDs), optical fibers, non-fiber optic waveguides, and/or lenses. Additionally, for example, the light source 138 can include a LED printed circuit board having one or more light sources (e.g., LEDs).

The optical structure 140 can be at a distal end of the shaft 126. In some examples, the optical structure 140 can circumferentially surround the electrosurgical electrode 128 to emit the light distally around all sides of the electrosurgical electrode 128. This can help to mitigate shadows and provide greater uniformity of illumination in all rotational alignments of the shaft 126 relative to the housing 123 and/or the electrosurgical device 112 relative to the target tissue. However, in other examples, the optical structure 140 can extend partially but not fully around the electrosurgical electrode 128.

In implementations that include the light source 138, the user input device(s) 130, the printed circuit board 132, the switches 136, and/or the housing conductor(s) 134 can additionally supply an electrical power from a direct current (DC) power source 142 to the light source 138. In one example, the DC power source 142 can include a battery disposed in the handle 124, the plug of the power cord 122, and/or a battery receptacle located along the power cord 122 between the handle 124 and the plug. Although the electrosurgical device 112 includes the DC power source 142 in FIG. 1, the DC power source 142 can be separate and distinct from the electrosurgical device 112 in other examples. For instance, in another example, the electrosurgical generator 110 can include the DC power source 142.

Additionally, in implementations that include the light source 138, the user input device(s) 130 can be operable to cause the light source 138 to emit the light. In one example, the user input device(s) 130 can include a button that independently controls the light source 138 separate from the button(s) that control the electrosurgical operational modes of the electrosurgical device 112. In another example, the user input device(s) 130 and the printed circuit board 132 can be configured such that operation of the button(s) that control the electrosurgical operational mode simultaneously control operation of the light source 138 (e.g., the light source 138 can be automatically actuated to emit light when a button is operated to apply the electrosurgical energy at the electrosurgical electrode 128).

As shown in FIG. 1, responsive to operation of the user input device(s) 130 to actuate the light source 138, the DC power source 142 can supply the electrical power (e.g., a DC voltage) to the light source 138 via the printed circuit board 132 and/or the housing conductor(s) 134. In this implementation, one or more of the conductive elements of the housing conductor(s) 134 can be configured to supply the electrical power from the DC power source 142 to the light source 138 and/or return the electrical power from the light source 138 to the DC power source 142. Accordingly, the housing conductor(s) 134 can additionally or alternatively assist in providing electrical communication between the DC power source 142 and the light source 138 as the shaft 126 and the light source 138 telescopically move relative to the handle 124.

Although the user input device(s) 130 on the handle 124 can be operated to control the operation of the light source 138 in the examples described above, the light source 138 can be additionally or alternatively operated by one or more user input device(s) on the electrosurgical generator 110 (e.g., via the user interface 116) and/or on the plug of the power cord 122.

In some examples, the electrosurgical device 112 can additionally or alternatively include features that provide for evacuating surgical smoke from a target tissue to a location external to the surgical site. Surgical smoke is a by-product of various surgical procedures. For example, during surgical procedures, surgical smoke may be generated as a by-product of electrosurgical units (ESU), lasers, electrocautery devices, ultrasonic devices, and/or other powered surgical instruments (e.g., bones saws and/or drills). In some instances, the surgical smoke may contain toxic gases and/or biological products that result from a destruction of tissue. Additionally, the surgical smoke may contain an unpleasant odor. For these and other reasons, many guidelines indicate that exposure of surgical personnel to surgical smoke should be reduced or minimized.

To reduce (or minimize) exposure to surgical smoke, a smoke evacuation system may be used during the surgical procedure. In general, the smoke evacuation system may include a suction pump 144 that can generate sufficient suction and/or vacuum pressure to draw the surgical smoke away from the surgical site. In some implementations, the smoke evacuation system may be coupled to an exhaust system (e.g., an in-wall exhaust system) that exhausts the surgical smoke out of an operating room. In other implementations, the smoke evacuation system may filter air containing the surgical smoke and return the air to the operating room. Within examples, the suction pump 144 and the electrosurgical generator 110 can be provided as separate devices or integrated in a single device (e.g., in a common housing).

As shown in FIG. 1, the shaft 126 can include a smoke evacuation channel 146 in the inner cavity 149 of the shaft 126. The smoke evacuation channel 146 can also include one or more smoke inlets at one or more positions around the electrosurgical electrode 128. In some examples, the smoke evacuation channel 146 can include a plurality of smoke inlets on opposing sides of the electrosurgical electrode 128. In this arrangement, the smoke inlet of the smoke evacuation channel can help to receive surgical smoke into the smoke evacuation channel 146 in a plurality of rotational alignments of the electrosurgical electrode 128 relative to the handle 124 and/or the electrosurgical device 112 relative to the target tissue.

In an example, the smoke evacuation channel 146 of the shaft 126 defines a first portion of a smoke flow path, and an interior bore 148 of the handle 124 defines a second portion of a smoke flow path. In this arrangement, the surgical smoke can be received from the surgical site into the smoke evacuation channel 146 of the shaft 126, and flow proximally along the smoke evacuation channel 146 to the interior bore 148 of the handle 124. In the interior bore 148 of the handle 124, the smoke can further flow to a smoke tube 150 that is coupled to a proximal end of the handle 124 and configured to convey smoke from the handle 124 to the suction pump 144.

As noted above, the electrosurgical electrode 128 can apply the electrosurgical energy to a target tissue to perform an electrosurgical operation (e.g., cutting, coagulating, ablating, and/or sealing the target tissue). Within examples, the electrosurgical electrode 128 can include an electrosurgical substrate formed from an electrically conductive material. As an example, the electrically conductive material can be stainless steel.

Additionally, as shown in FIG. 1, the electrosurgical device 112 can include a camera 152 that is configured to capture one or more images, and the electrosurgical system 100 can include a display device 154 that is configured to display the one or more images captured by the camera 152. The camera 152 can be communicatively coupled to a display device 154 (e.g., via a wired or wireless network, e.g., LAN, WAN, Internet, cloud, near-field communications, etc.). In this arrangement, the camera 152 can communicate the one or more images to the display device 154, and the display device 154 can display the one or more images to a practioner that is operating the electrosurgical device 112.

In some examples in which the camera 152 is coupled to the display device by a wired connection, the electrosurgical device 112 can include one or more wires that extend through the housing 123. For instance, the one or more wires can extend through and from a proximal end of the housing 123. In some such implementations, the one or more wires can be bundled with the power cord 122 in a common casing (e.g., a heat shrink sleeve). This can help to enhance cable management and reduce tangling of the one or more wires with other cables in an operational environment. In other examples, the one or more wires can extend from directly from the camera 152 to the display device 154 and outside of the housing 123. This may be beneficial in implementations in which the electrosurgical device 112 is retrofit with the camera 152

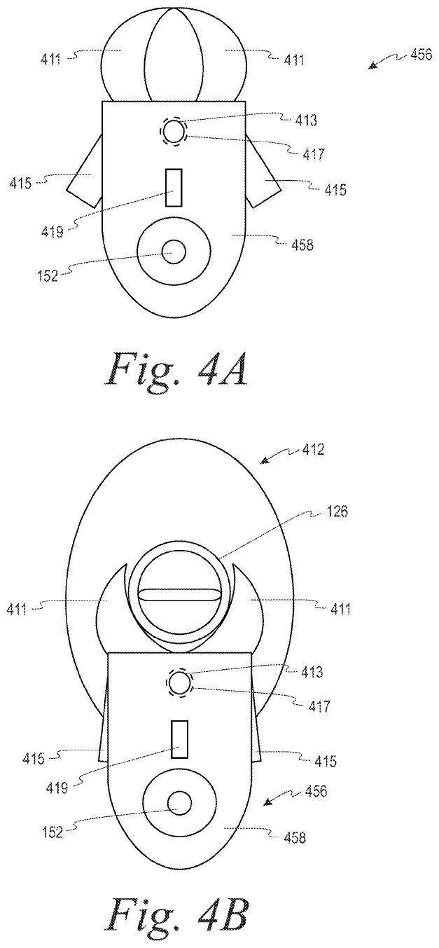

As described in further detail below with respect to FIGS. 4-7, the electrosurgical device 112 can include a wireless transmitter that is communicatively coupled to the camera 152 and configured to communicate the one or more images to the display device 154. For instance, in some implementations, the electrosurgical device 112 can include a wireless transmitter that can communicate the one or more images to a wireless receiver that is operatively coupled to the display device 154. This can help to mitigate cable management issues and/or improve handling of the electrosurgical device 112 by reducing a quantity and/or thickness of a cable extending from the proximal end of the electrosurgical device 112 (e.g., including the power cord 122).

In some examples, the camera 152 can be configured to capture the one or more images and transmit the one or more images as still images and/or as video. Capturing and/or displaying still images may be helpful to obtain pre-operative and/or post-operative pictures for analysis of the electrosurgical procedure. Capturing and/or displaying video using the camera 152 can additionally or alternatively help to increase visibility of the electrosurgical electrode 128, a target tissue, and/or the surgical site.

Within examples, the camera 152 can capture the one or more images while supplying electrosurgical energy to the electrosurgical electrode 128. This can allow for the camera 152 and the display device 154 to facilitate displaying the one or more images in real time during the electrosurgical procedure (e.g., while performing a cutting operation and/or a coagulating operation). Additionally, within examples, the light source 138 can be configured to generate light and illuminate (e.g., via the optical structure 140), the electrosurgical electrode 128, the target tissue, and/or the surgical site while the camera 152 captures the one or more images. As such, a field of view of the camera 152 can at least partially overlap with an illumination area of the light output by the light source 138 and/or the optical structure 140.

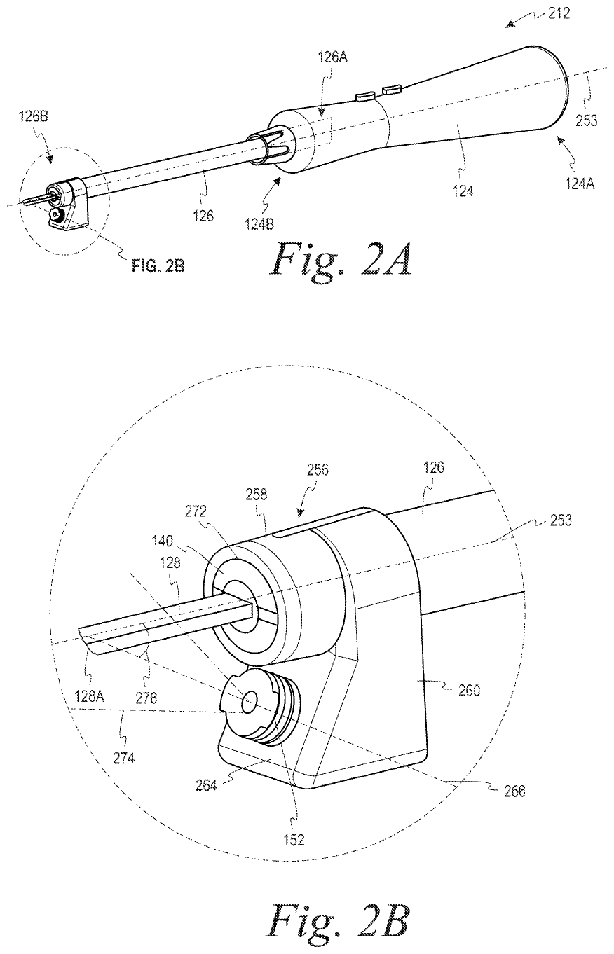

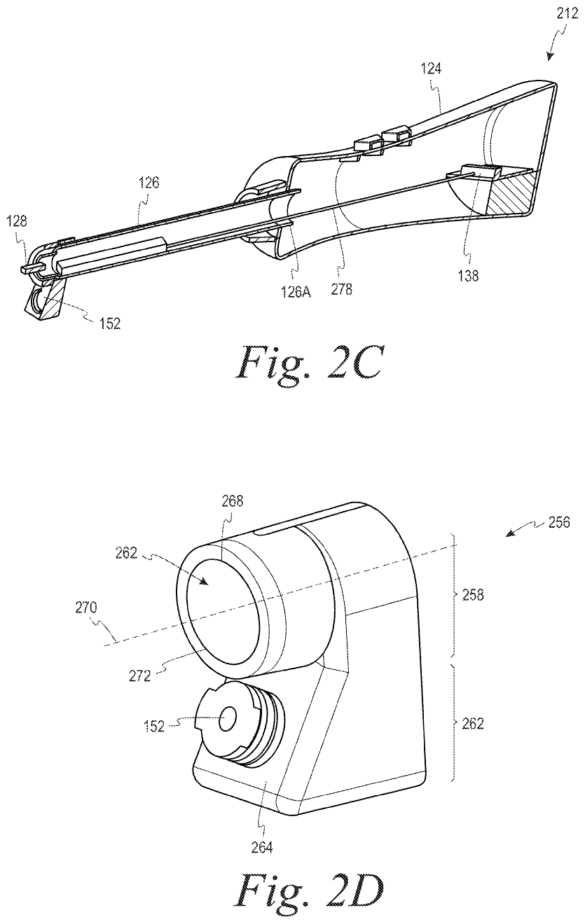

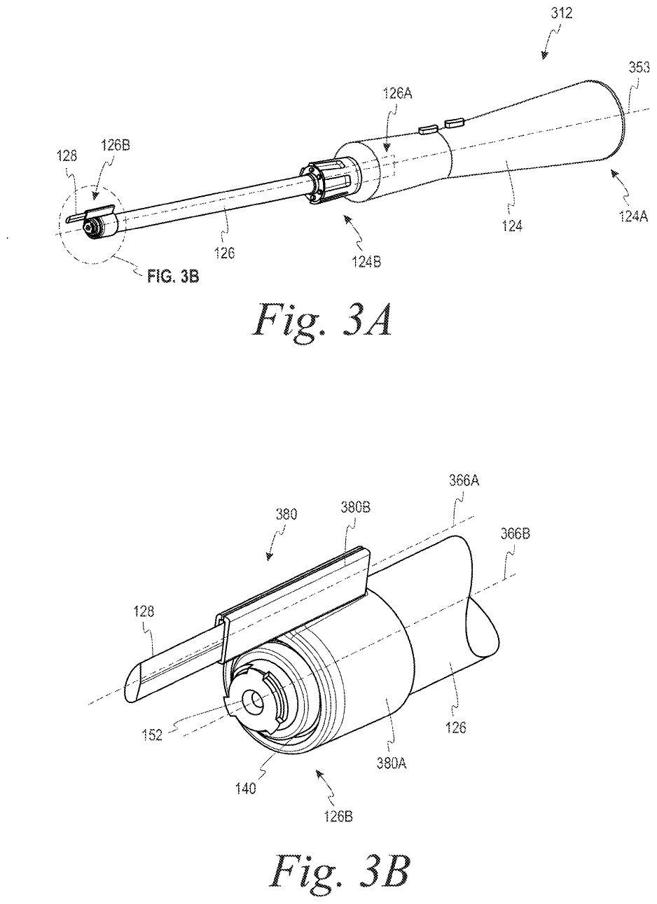

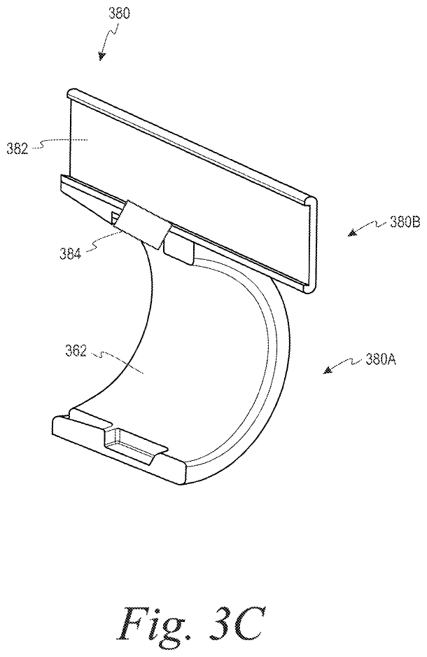

Within examples, the camera 152 can be configured with a field of view that is directed in a distal direction to capture the one or more images of at least a portion of the electrosurgical electrode 128, a target tissue, and/or a surgical site. This can help a practioner to better visualize a spatial relationship between the electrosurgical electrode 128 and the target tissue and/or non-target tissue at the surgical site, and/or an effect of the electrosurgical energy on the target tissue and/or non-target tissue at the surgical site. Example implementations for coupling the camera 152 to the housing 123 (e.g., the shaft 126 and/or the handle 124) such that the field of view that is directed in a distal direction to capture the one or more images of at least a portion of the electrosurgical electrode 128, a target tissue, and/or a surgical site are described below with respect to FIGS. 2A-3C.

In some examples, the camera 152 can be coupled to the housing 123 such that the field of view of the camera 152 is fixed and non-adjustable relative to the housing 123. This can help to simplify the coupling of the camera 152 and the housing 123, which can lead to (i) reduced costs, (ii) simplified manufacturing, (iii) simplified assembly, (iv) reduced weight, and/or (v) reduced size which can improve line of sight. In other examples, the camera 152 can be coupled to the housing 123 such that the field of view of camera 152 is adjustable relative to the housing 123 while the camera 152 is coupled to the housing 123. For instance, in some implementations, the camera 152 can be coupled to the housing 123 by a

In some examples, the camera 152 can be operatively coupled to the DC power source 142. For instance, in FIG. 1, the camera 152 is coupled to the DC power source 142 by the PCB 132. However, in other examples, the camera 152 can be directly coupled to the DC power source 142 separately from the PCB 132 (or the PCB 132 can be entirely omitted). In examples in which the camera 152 is operatively coupled to the DC power source 142, the camera 152 can use the DC power provided by the DC power source 142 to capture the one or more images of an area of interest.

In other examples, the electrosurgical device 112 can omit the DC power source 142, and the camera 152 can receive power from the power cord 122. In such examples, the PCB 132 can include a power converter to convert a portion of the electrosurgical energy into a suitable power supply for the camera 152.

Referring now to FIGS. 2A-2C, an electrosurgical device 212 is shown according to one example implementation for the electrosurgical device 112 described above, In particular, FIG. 2A depicts a perspective view of the electrosurgical device 212, FIG. 2B depicts an enlarged view of a distal portion of the electrosurgical device 212 shown in FIG. 2A, and FIG. 2C depicts a partial cross-sectional view of the electrosurgical device 212 (omitting certain components for ease of illustration of other components) through a longitudinal axis 253 according to an example.

The electrosurgical device 212 is substantially similar or identical to the electrosurgical device 112, described above. As shown in FIGS. 2A-2C, the electrosurgical device 212 includes the handle 124 and the shaft 126. The handle 124 extends between a proximal handle end 124A and a distal handle end 124B, and the shaft 126 extends from the distal handle end 124B of the handle 124. The shaft 126 extends between a proximal shaft end 126A and a distal shaft end 126B. The electrosurgical electrode 128 extends from the distal shaft end 126B.

In the example shown in FIG. 2A, the shaft 126 is telescopically moveable and/or rotatable relative to the handle 124. As described above, this can provide for adjusting a length of the electrosurgical device 112, which can facilitate performing electrosurgery at a plurality of different depths within tissue (e.g., due to different anatomical shapes and/or sizes of patients) and/or at a plurality of different angles. In other examples, the shaft 126 and/or the electrosurgical electrode 128 can be axially fixed and/or non-rotatable relative to the handle 124.

Additionally, in this example, the electrosurgical electrode 128 is coupled to the shaft 126. This can provide for the electrosurgical electrode 128 moving axially and/or rotating with the shaft 126 relative to the handle 124. In other examples, the electrosurgical electrode 128 can be coupled to the handle 124, and/or the electrosurgical electrode 128 can be movable and/or rotatable independently of the shaft 126.

As shown in FIGS. 2A-2C, the electrosurgical device 212 includes a camera assembly 256 coupled to the distal shaft end 126B. FIG. 2D also depicts a perspective view of the camera assembly 256 shown in FIGS. 2A-2C, according to this example. As shown in FIGS. 2A-2D, the camera assembly 256 includes (i) a first housing portion 258 that is configured to couple to the shaft 126, and (ii) a second housing portion 260 that extends laterally outward from the first housing portion 258 and the shaft 126, and includes the camera 152.

For instance, as shown in FIGS. 2A-2D, the first housing portion 258 can include a through-bore 262 receiving the distal shaft end 126B to couple the camera assembly 256 to the shaft 126. Additionally, as shown in FIGS. 2A-2D, the second housing portion 260 can extend laterally outward from the first housing portion 258 and the shaft 126, and the second housing portion 260 can include a camera mount surface 264 extending in a plane that has a normal line 266 extending toward the electrosurgical electrode 128. The camera 152 is coupled to the camera mount surface 264. In this way, the first housing portion 258 can couple the camera assembly 256 to the shaft 126, and the second housing portion 260 can arrange the camera 152 such that the field of view of the camera 152 can include the electrosurgical electrode 128, the target tissue, and/or the surgical site.

Within examples, the first housing portion 258 can be coupled to the shaft 126 by at least one coupling selected from a group consisting of: a friction-fit coupling, an adhesive coupling, a threaded coupling, a snap-fit coupling, an ultrasonic weld coupling, and an over-molding coupling. In some examples, the first housing portion 258 can be configured to rotate about the shaft 126 while the distal shaft end 126B is received in the through-bore 262. For instance, in one implementation, the first housing portion 258 can include a protrusion 268 that extends inwardly from an interior surface of the first housing portion 258 towards a center axis 270 of the through-bore 262, and the shaft 126 can include a recess that can receive the protrusion 268. In this arrangement, the protrusion and the recess can allow for rotation of the first housing portion 258 relative to the shaft 126 while inhibiting axial movement of the first housing portion 258 relative to the shaft 126. In another implementation, the first housing portion 258 can include the recess and the shaft 126 can include the protrusion, which can extend from the shaft towards the first housing portion 258 and be received in the recess of the shaft 126. Rotation of the first housing portion 258 relative to the shaft 126 can help to rotate the field of view of the camera 152. This may help to improve access to the surgical site, and/or improve a direct line of sight of the surgical site around the camera assembly 256.

In other examples, the first housing portion 258 can be non-rotationally fixed relative to the shaft 126. This may simplify manufacture and/or operation of the electrosurgical device 212. Additionally, in implementations in which the shaft 126 is rotatable relative to the handle 124, rotation of the camera 152 relative to the handle 124 can be achieved by rotating the shaft 126 relative to the handle 124.

As shown in FIGS. 2A-2D, first housing portion 258 can include an aperture 272 at a distal end of the camera assembly 256. As shown in FIG. 2B, the electrosurgical electrode 128 extends distally through the aperture 272 at the distal end of the camera assembly 256. As such, the aperture 272 can have a size that is greater than a size of the electrosurgical electrode 128 at the distal end of the camera assembly 256. This can allow the electrosurgical electrode 128 to be exposed and extend distally of the distal shaft end 126B and the camera assembly 256 such that the electrosurgical electrode 128 can perform an electrosurgical operation on the target tissue.

In some examples, the electrosurgical device 212 can additionally or alternatively include the light source 138 and/or the optical structure 140 as described above. As shown in FIG. 2B, the light source 138 and/or the optical structure 140 can be configured to emit light through the aperture 272 at the distal end of the camera assembly 256. This can provide for the light source 138 and/or the optical structure 140 emitting the light in a distal direction (e.g., along a length of the electrosurgical electrode 128), which can help to better visualize the electrosurgical electrode 128, the target tissue, and/or the surgical site by direct visualization and/or by the one or more images captured by the camera 152.

In FIG. 2B, the optical structure 140 extends around a circumference of the electrosurgical electrode 128. This can help to emit the light distally around all sides of the electrosurgical electrode 128, which can help to mitigate shadows and provide greater uniformity of illumination in all rotational alignments of the shaft 126 relative to the housing 123 and/or the electrosurgical device 112 relative to the target tissue. However, in other examples, the optical structure 140 can extend partially but not fully around the electrosurgical electrode 128.

Additionally, in other examples, the electrosurgical device 212 can omit the optical structure 140 and instead include the light source 138 the position shown for the optical structure 140 in FIG. 2B (e.g., in the first housing portion 258 and at the aperture 272). In such examples, the light source 138 can directly emit the light through the aperture 272 instead of emitting the light through the aperture 272 via the optical structure 140.

As shown in FIGS. 2B and 2D, the aperture 272 at the distal end of the camera assembly 256 can be coaxial with the longitudinal axis 253 of the electrosurgical electrode 128 and the longitudinal axis 253 of the shaft 126. In implementations that include the light source 138 and/or the optical structure 140 surrounding the electrosurgical electrode 128, this can help to provide substantially uniform illumination around the electrosurgical electrode 128. In other implementations, the aperture 272 can additionally or alternatively provide an inlet to the smoke evacuation channel 146 of the shaft 125. In such implementations, configuring the aperture 272 to be coaxial with the longitudinal axis 253 of the electrosurgical electrode 128 and the longitudinal axis 253 of the shaft 126 can help to provide substantially uniform suction around the electrosurgical electrode 128.

In still other examples, the electrosurgical device 212 can omit the light source 138, the optical structure 140, and the smoke evacuation channel 146. In such examples, the aperture 272 can have a size and a shape that matches a cross-sectional size and shape of the electrosurgical electrode 128 such that a liquid-tight seal is provided at the distal end of the camera assembly 256. This can help to mitigate ingress of fluids (e.g., blood or irrigation fluids) into the camera assembly 256.

As described above, the second housing portion 260 can include the camera mount surface 264 extending in the plane that has the normal line 266 extending toward the electrosurgical electrode 128, and the camera 152 is coupled to the camera mount surface 264. In some examples (e.g., as shown in FIG. 2B), the camera mount surface 264 and the camera 152 coupled to the camera mount surface 264 are configured such that a field of view 274 of the camera 152 includes a distal tip 128A of the electrosurgical electrode 128. This can help to enhance visibility of the electrosurgical electrode 128, a target tissue, and/or a surgical site relative to alternative implementations in which the camera is mounted in-line with the electrosurgical electrode 128. In some examples, the camera mount surface 264 and the camera 152 coupled to the camera mount surface 264 are configured such that a field of view 274 of the camera 152 includes the distal tip 128A of the electrosurgical electrode 128 and an environment that is distal of the distal tip 128A. In some examples, the camera mount surface 264 and the camera 152 coupled to the camera mount surface 264 are configured such that a field of view 274 of the camera 152 includes the distal tip 128A and at least 40 percent of a length of the electrosurgical electrode 128. This can help to provide for visualization of a working portion of the electrosurgical electrode 128 in many implementations (e.g., in an implementation in which a proximal portion of the electrosurgical electrode 128 is covered by a heat shrink, insulator material). In other implementations, the camera mount surface 264 and the camera 152 coupled to the camera mount surface 264 are configured such that a field of view 274 of the camera 152 includes the distal tip 128A and different percentages of a length of the electrosurgical electrode 128 In some examples, an angle 276 between the normal line 266 of the plane of the camera mount surface 264 and the longitudinal axis 253 of the shaft 126 can be between approximately 20 degrees and approximately 30 degrees. This can help to couple the camera 152 to the camera mount surface 264 such that a principal axis of the camera 152 can be collinear with the normal line 266 and achieve the field of view 274 described above. In another example, the angle 276 can be between approximately 23 degrees and approximately 27 degrees. This can help to reduce an overall bulk of the distal shaft end 126B, keep the camera 152 closer to the electrosurgical electrode 128 so that overall accessibility can be improved (e.g., the larger the angle 276, the more difficult it gets to access tight spaces in some instances). Although the example angle 276 described above can be beneficial, the angle 276 can be different in other examples.

As shown in FIGS. 1 and 2C, the camera 152 can be communicatively coupled with the PCB 132 in some examples. In the example shown in FIG. 2C, the PCB 132 is located in the handle 124, and the PCB 132 is coupled to the camera 152 by one or more camera signal wires 278. However, in other examples, the PCB 132 can be located in the shaft 126 and/or the camera assembly 256. In other examples, the camera 152 can be configured to communicate the one or more images to the display device 154 independently of the PCB 132.

Referring now to FIGS. 3A-3B, an electrosurgical device 312 is shown according to another example implementation for the electrosurgical device 112 described above. In particular, FIG. 3A depicts a perspective view of the electrosurgical device 312, and FIG. 3B depicts an enlarged view of a distal portion of the electrosurgical device 312 shown in FIG. 3A, according to an example.

The electrosurgical device 312 is substantially similar or identical to the electrosurgical device 112, described above. As shown in FIGS. 3A-3B, the electrosurgical device 312 includes the handle 124 and the shaft 126. The handle 124 extends between a proximal handle end 124A and a distal handle end 124B, and the shaft 126 extends from the distal handle end 124B of the handle 124. The shaft 126 extends between a proximal shaft end 126A and a distal shaft end 126B. The electrosurgical electrode 128 extends from the distal shaft end 126B.

In the example shown in FIG. 3A, the shaft 126 is telescopically moveable and/or rotatable relative to the handle 124. As described above, this can provide for adjusting a length of the electrosurgical device 112, which can facilitate performing electrosurgery at a plurality of different depths within tissue (e.g., due to different anatomical shapes and/or sizes of patients) and/or at a plurality of different angles. In other examples, the shaft 126 and/or the electrosurgical electrode 128 can be axially fixed and/or non-rotatable relative to the handle 124.

As shown in FIG. 3B, the electrosurgical device 312 includes the camera 152 at the distal shaft end 126B. In some examples, the camera 152 can be disposed in the inner cavity 149 of the shaft 126. In other examples, the camera 152 can be coupled to an exterior portion of distal shaft end 126B. Within examples, the camera 152 can be coupled to the shaft 126 by at least one coupling selected from a group consisting of: a friction-fit coupling, an adhesive coupling, a threaded coupling, a snap-fit coupling, an ultrasonic weld coupling, and an over-molding coupling.

As shown in FIGS. 3A-3B, the electrosurgical device 312 also includes the electrosurgical electrode 128 coupled to an exterior surface the shaft 126 and extending distally of the distal shaft end 126B. A longitudinal axis 366A of the electrosurgical electrode 128 can be offset from an axis 366B, which is a principal axis of the camera 152 and/or a longitudinal axis of the shaft 126. In this arrangement, the electrosurgical electrode 128 and a field of view of the camera 152 can both be directed generally in a distal direction (e.g., in a direction extending from the proximal shaft end 126A toward the distal shaft end 126B). This can help to provide a direct connection with a hand of a user as the user moves the electrosurgical device 112.

In some implementations, the field of view of the camera 152 can be sufficiently wide to provide visibility of the electrosurgical electrode 128. For example, the field of view can be between approximately 45 degrees and approximately 90 degrees. In another example, the field of view of can be between approximately 45 degrees and approximately 60 degrees. In another example, the field of view can be between approximately 70 degrees and 90 degrees. In still other examples, the field of view can have different angles.

In some examples, such as the example shown in FIGS. 3A-3B, the longitudinal axis 366A of the electrosurgical electrode 128 can also be parallel to the axis 366B (e.g., the longitudinal of the shaft 126 and/or the principal axis of the field of view of the camera 152). This can help to enhance visibility of the electrosurgical electrode 128 via direct visualization and/or via the one or more images captured by the camera 152.

As shown in FIG. 3B, the electrosurgical electrode 128 is coupled to the exterior surface of the shaft 126 by a collar 380. The collar 380 includes a first collar portion 380A that couples to the exterior surface of the shaft 126 at a distal portion of the shaft 126, and a second collar portion 380B that couples the electrosurgical electrode 128 to the first collar portion 380A.

In the example shown in FIG. 3B, the first collar portion 380A can include a through-bore 362 receiving the distal portion of the shaft 126. In some examples, the first collar portion 380A can be coupled to the shaft 126 by at least one coupling selected from a group consisting of: a friction-fit coupling, an adhesive coupling, a threaded coupling, a snap-fit coupling, an ultrasonic weld coupling, and an over-molding coupling. In some examples, the first collar portion 380A can be configured to rotate about the shaft 126 while the distal shaft end 126B is received in the through-bore 362. For instance, in one implementation, the first collar portion 380A can include a protrusion that extends inwardly from an interior surface of the first collar portion 380A towards a center axis of the through-bore 262 (e.g., the axis 366B in FIG. 3B), and the shaft 126 can include a recess that can receive the protrusion. In this arrangement, the protrusion and the recess can allow for rotation of the first collar portion 380A relative to the shaft 126 while inhibiting axial movement of the first collar portion 380A relative to the shaft 126. In another implementation, the first collar portion 380A can include the recess and the shaft 126 can include the protrusion, which can extend from the shaft towards the first collar portion 380A and be received in the recess of the shaft 126. Rotation of the first collar portion 380A relative to the shaft 126 can help to rotate the electrosurgical electrode 128 relative to the shaft 126 and the field of view of the camera 152. This may help to improve access to the surgical site, and/or improve visualization of the electrosurgical electrode 128, the target tissue, and/or the surgical site (e.g., via direct visualization and/or via the one or more images captured by the camera 152).

In other examples, the first collar portion 380A can be non-rotationally fixed relative to the shaft 126. This may simplify manufacture and/or operation of the electrosurgical device 312. Additionally, in implementations in which the shaft 126 is rotatable relative to the handle 124, rotation of the electrosurgical electrode 128 relative to the handle 124 can be achieved by rotating the shaft 126 relative to the handle 124.

As shown in FIG. 3B, the second collar portion 380B can define an elongated slot 382 for receiving a proximal portion of the electrosurgical electrode 128. FIG. 3C depicts a cross-sectional view of the collar 280 taken through the longitudinal axis 366A to further illustrate the slot 382. Within examples, the electrosurgical electrode 128 can be coupled to the second collar portion 380B in the slot 382 by at least one coupling selected from a group consisting of: a friction-fit coupling, an adhesive, a snap-fit coupling, an ultrasonic weld coupling, and an over-molding coupling.

Within examples, the electrosurgical electrode 128 can be electrically coupled to the shaft 126 such that electrosurgical energy is supplied from the electrosurgical generator 110 to the electrosurgical electrode 128 via the shaft 126. For instance, as shown in FIG. 3C, the collar 380 can include a conductor 384 that extends from the through-bore 362 of the first collar portion 380A to the elongated slot 382 of the second collar portion 380B to electrically couple the electrosurgical electrode 128 to the shaft 126. In other examples, the shaft 126 and/or the collar 380 can include one or more apertures for the housing conductor 134 to extend through and couple to the electrosurgical electrode 128, which is positioned in the elongated slot 382 of the second collar portion 380B.

In some examples, the electrosurgical device 312 can additionally or alternatively include the light source 138 and/or the optical structure 140 as described above. As shown in FIG. 3B, the light source 138 and/or the optical structure 140 can be configured to emit light at the distal shaft end 126B. This can provide for the light source 138 and/or the optical structure 140 emitting the light in a distal direction (e.g., along a length of the electrosurgical electrode 128), which can help to better visualize the electrosurgical electrode 128, the target tissue, and/or the surgical site by direct visualization and/or by the one or more images captured by the camera 152.

In FIG. 3B, the optical structure 140 extends around a circumference of the camera 152. This can help to emit the light distally around all sides of the camera 152, which can help to mitigate shadows and provide greater uniformity of illumination in all rotational alignments of the shaft 126 relative to the housing 123 and/or the electrosurgical device 312 relative to the target tissue. However, in other examples, the optical structure 140 can extend partially but not fully around the electrosurgical electrode 128.

Additionally, in other examples, the electrosurgical device 312 can omit the optical structure 140 and instead include the light source 138 at the position shown for the optical structure 140 in FIG. 3B. In such examples, the light source 138 can directly emit the light from the distal shaft end 126B instead of emitting the light via the optical structure 140.

FIGS. 4A-4B depict another implementation of a camera assembly 456 according to another example. FIG. 4A depicts a front view of the camera assembly 456, and FIG. 4B depicts the camera assembly of FIG. 4A coupled to the shaft 126 of an electrosurgical device 412. The electrosurgical device 412 is substantially similar or identical to the electrosurgical device 112, described above. In this example, the camera assembly 456 includes a spring-clip mechanism for coupling the camera assembly 456 to the shaft 126 of the electrosurgical device 412.

For instance, as shown in FIG. 4A, the camera assembly 456 can include a camera housing 458, and the camera 152 is coupled to a distal surface of the camera housing 458. The camera assembly 456 also includes a pair of jaws 411 that are hingedly coupled to the camera housing 458 at a pivot point 413 (e.g., a pin). The jaws 411 are configured to be actuated between a closed position and an open position by one or more handle buttons 415. The jaws 411 can be biased towards the closed position by a spring 417. The handle buttons 415 can be configured to be moved towards the camera housing 458 with a force that exceeds a biasing force applied to the jaws 411 by the spring 417 to actuate the jaws 411 from the closed position to the open position.

When the jaws 411 are moved from the closed position towards the open position, a space between the jaws 411 increases in size. When the jaws 411 are moved from the open position towards the closed position, the space between the jaws 411 decreases in size. In this arrangement, responsive to operating the handle buttons 415 to actuate the jaws 411 towards the open position, the shaft 126 can be positioned between the jaws 411. Then, responsive to releasing the handle buttons 415, the spring 417 can cause the jaws 411 to move from the open position toward the closed position and clamp onto the shaft 126 of the electrosurgical device 412 as shown in FIG. 4B.

In some examples, the biasing force applied by the spring 417 to the jaws 411 can be suitable to inhibit (or prevent) the camera assembly 456 from moving relative to the shaft 126 while performing an electrosurgical operation. In other examples, the camera assembly 456 can additionally or alternatively include a releasable lock 419 that can help to mitigate movement of the camera assembly 456. For instance, the releasable lock 419 can have a locked state and an unlocked position. In the locked state, the releasable lock 419 can prevent the jaws 411 from moving toward the open position. In the unlocked state, the releasable lock 419 can allow the jaws to move from toward the open state. As shown in FIGS. 4A-4B, at least a portion of the releasable lock 419 can be operable on an exterior of the camera housing 458 to actuate the releasable lock 419 between the unlocked state and the locked state.

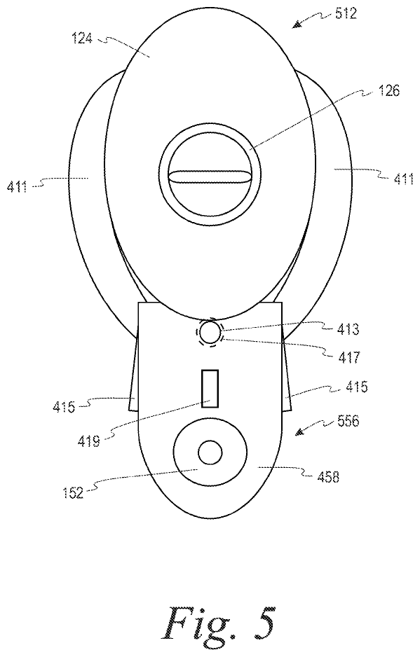

FIG. 5 depicts another implementation of a camera assembly 556 coupled to an electrosurgical device 512, according to another example. The camera assembly 556 is identical to the camera assembly 456 described above with respect to FIGS. 4A-4B, except the jaws 411 of the camera assembly 556 are configured to clamp onto the handle 124 of the electrosurgical device 512. The electrosurgical device 512 is substantially similar or identical to the electrosurgical device 112, described above.

As described above, the camera 152 can be coupled to the shaft 126 and/or the handle 124 within examples. FIGS. 4A-5 depict implementations that can provide for coupling the camera 152 to an exterior surface of the shaft 126 and/or the handle 124. Accordingly, in some implementations, the practioner can select a position for the camera 152 on the shaft 126 and/or the handle 124 from among a plurality of potential axial positions and/or radial positions on the shaft 126 and/or the handle 124 such that position of the camera 152 can be continuously selected over the shaft 126 and/or the handle 124. This can provide for greater flexibility and/or user preference in selecting a position on the shaft 126 and/or the handle 124 to mount the camera 152.

In other examples, the handle 124 and/or the shaft 126 can include one or more discrete mounting positions for coupling to the camera 152. In such examples, the camera 152 can only be coupled to the handle 124 and/or the shaft 126 at specific, predefined positions along the handle 124 and/or the shaft 126. For instance, the handle 124 and/or the shaft 126 can include a first coupling structure, a camera assembly can include a second coupling structure, and the first coupling structure can be configured to couple to the second coupling structure. As examples, the first coupling structure and the second coupling structure can be configured to couple to each other by at least one mechanism selected from a group consisting of: (i) a threaded coupling, (ii) a quick-release coupling, (iii) a magnetic coupling, (iv) a cold shoe and hot shoe coupling, (v) a ball-and-socket joint, (vi) a dovetail mount, and (vii) a bayonet mount coupling.

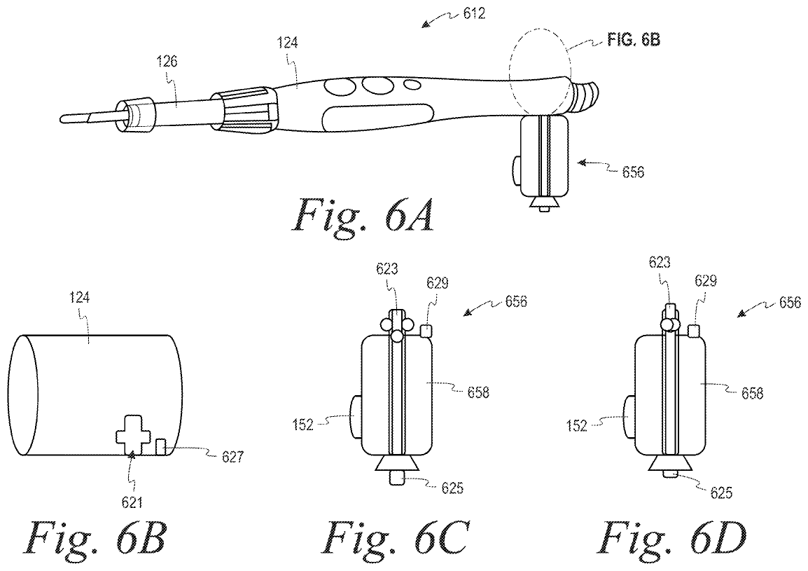

As one example implementation, FIGS. 6A-6D depict an electrosurgical device 612 and a camera assembly 656 that are configured to be coupled to each other by a quick-release coupling, according to an example. FIG. 6A depicts the camera assembly 656 coupled to the handle 124 of the electrosurgical device 612, FIG. 6B depicts a portion of the handle 124 including the quick-release coupling, FIG. 6C depicts the camera assembly 656 in a first state, and FIG. 6D depicts the camera assembly 656 in a second state. The electrosurgical device 612 is substantially similar or identical to the electrosurgical device 112, described above.

In this example, the handle 124 of the electrosurgical device 612 includes a slot 621 and the camera assembly 656 includes a post 623 having one or more spring-loaded balls. The camera assembly 656 also includes an actuator 625 on a camera housing 658 that can be operated to move the spring-loaded ball(s) inwardly. FIG. 6C shows the camera assembly 656 in the first state in which the actuator 625 is in an extended position and the spring-loaded balls are in an outwardly extending position. FIG. 6D shows the camera assembly 656 in the second state in which the actuator 625 is in a depressed position and the spring-loaded balls are in an inwardly retracted position. In this arrangement, the post 623 can be inserted into the slot 521, and one or more side channels of the slot 621 can receive the spring-loaded ball(s) to axially retain the post 623 in the slot 621. The actuator 625 can be operated to move the spring-loaded ball(s) out of the side channel(s) of the slot 621 to allow the post 623 to be removed from the slot 621.

In FIGS. 6B-6D, the handle 124 can also include a first power connector 627 and the camera assembly 656 can include a second power connector 629 that can electrically couple to the first power connector 627 when the camera assembly 656 is coupled to the handle 124. The coupling between the first power connector 627 and the second power connector 629 can provide for supplying power to the camera 152 and/or communicating data (e.g., images) from the camera 152 to a display device and/or a controller (e.g., the controller 141). In FIGS. 6B-6D, the first power connector 627 is a receptacle and the second power connector 629 is a plug, but the first power connector 627 and the second power connector 629 can be configured differently in other examples.

Although the electrosurgical device 612 includes the slot 621 at a single location on the handle 124 in FIG. 6A, the electrosurgical device 612 can include one or more slots 621 at one or more additional or alternative locations on the handle 124 and/or the shaft 126 in other examples. In some implementations that include a plurality of slots 621, a plurality of camera assemblies 656 can be coupled to the electrosurgical device 612 at the same time to provide a plurality of fields of view and/or to facilitate image processing, described in further detail below.

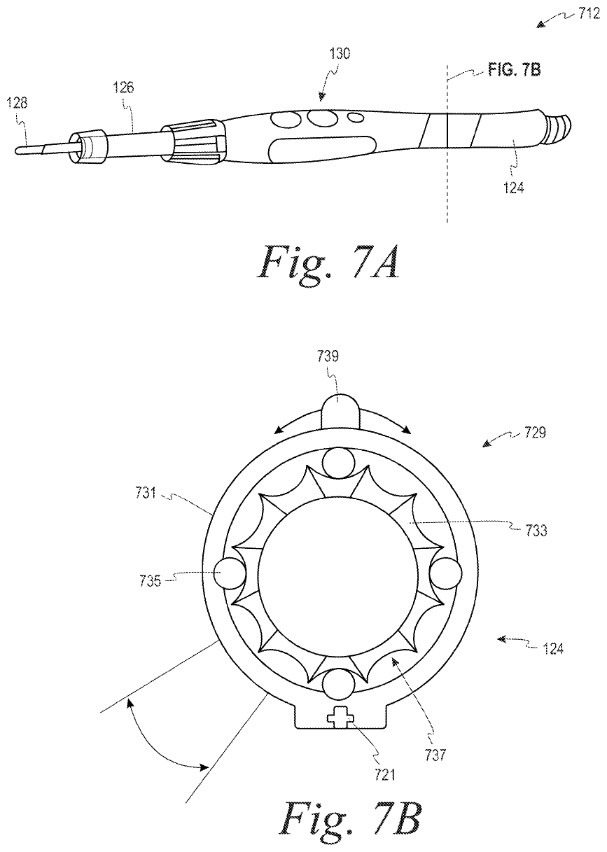

In some examples, the electrosurgical device 112 can include one or more features that provide for rotation of the camera 152 around a circumference of the shaft 126 and/or the handle 124 (e.g., rotation about the longitudinal axis of the shaft 126 and/or the handle 124). FIG. 7A depicts an electrosurgical device 712, and FIG. 7B depicts a cross-sectional view of the handle 124 of the electrosurgical device 712 taken through a line in shown in FIG. 7A, according to an example.

The electrosurgical device 712 can be substantially similar or identical to the electrosurgical device 112, 612 described above. As shown in FIGS. 7A-7B, the shaft 126 and/or the handle 124 of the electrosurgical device 712 includes a first coupling structure 721 that is configured to couple to the second coupling structure of a camera assembly (e.g., the camera assembly 656), as described above. Although FIGS. 7A-7C depict the first coupling structure 721 implemented as the slot shown in FIGS. 6A-6D, the first coupling structure 721 can be configured to couple to the second coupling structure of the camera assembly by at least one mechanism selected from a group consisting of: (i) a threaded coupling, (ii) a quick-release coupling, (iii) a magnetic coupling, (iv) a cold shoe and hot shoe coupling, (v) a ball-and-socket joint, (vi) a dovetail mount, and (vii) a bayonet mount coupling, as described above.

As shown in FIGS. 7A-7B, the electrosurgical device 712 can include a rotatable mount assembly 729 that provides an indexed bearing mechanism to facilitate rotation of the first coupling structure 721 of the electrosurgical device 712 relative to a non-rotatable portion of the electrosurgical device 712 (e.g., a portion of the shaft 126, a portion of the handle 124, the electrosurgical electrode 128, and/or the user input device(s) 130).

As shown in FIGS. 7B-7C, the rotatable mount assembly 729 can include an exterior housing portion 731, an internal support structure 733 disposed within a housing lumen of the exterior housing portion 731, and a plurality of ball bearings 735 disposed between an outer surface of the internal support structure 733 and an inner surface of the exterior housing portion 731. The internal support structure 733 can be fixed and non-rotationally disposed in the housing 123, whereas the exterior housing portion 731 can be rotatably coupled to the internal support structure 733 by the ball bearings 735. In this arrangement, with the first coupling structure 721 disposed on the exterior housing portion 731, the first coupling structure 721 and a camera assembly coupled to the first coupling structure 721 can rotate relative to the non-rotational portion of the electrosurgical device 712.

In this example, the ball bearings 735 are retained within respective recesses on the inner surface of the exterior housing portion 731. The recesses can be, for instance, hemispherical shaped recesses, and at least a portion of each ball bearing 735 protrudes from the respective recess in which the ball bearing 735 is disposed.

As shown in FIGS. 7B-7C, in some examples, the outer surface of the internal support structure 733 can have a plurality of grooves 737 that are each configured to receive a respective one of the ball bearings 735. The grooves 737 can define discrete, rotational positions of the first coupling structure 721 on the electrosurgical device 712. In the implementation shown in FIG. 7B, the grooves 737 can be separated by 30 degree intervals around a circumference of the housing 123. This can provide for twelve discrete rotational positions of the first coupling structure 721 (and the camera 152) around the electrosurgical device 712. In other implementations, the internal support structure 733 can a different quantity of grooves (e.g., the grooves 737 can be separated by 15 degree intervals, 45 degree intervals, 60 degree intervals, 90 degree intervals, or 120 degree intervals).

The grooves 737 and the ball bearings 735 can be configured to inhibit inadvertent rotation of the exterior housing portion 731 relative to the internal support structure 733. For instance, in the implementation of FIGS. 7A-7B, the ball bearings 735 and the grooves 737 can be sized such that a threshold amount of force is required to be applied to advance the ball bearings 735 from one set of grooves 737 to adjacent ones of the grooves 737. The threshold amount of force can be greater than a force that is applied to the exterior housing portion 731 by a weight of the camera assembly coupled to the first coupling structure 721. This can help to to mitigate inadvertent rotation of the camera assembly.

As shown in FIG. 7B, the exterior housing portion 731 can optionally include a knob 739 that extends outwardly from the exterior housing portion 731. The knob 739 can help to better grip and rotate the exterior housing portion 731.

Additionally, as shown in FIG. 7B, the internal support structure 733 can define an interior lumen 741. The interior lumen 741 can provide space for housing and/or passing through other components of the electrosurgical device 712 described above with respect to FIG. 1.

In some examples, the rotatable mount assembly 729 can also include a locking mechanism to further inhibit inadvertent rotation of the exterior housing portion 731 and the first coupling structure 721. For instance, in some examples, the exterior housing portion 731 can be axially movable relative to internal support structure 733 between a locked position and an unlocked position. In these examples, the grooves 737 of the internal support structure 733 and the ball bearings 735 can be configured to prevent rotation of the exterior housing portion 731 when the exterior housing portion 731 is in the locked position. When the exterior housing portion is in the unlocked position, the ball bearings 735 can be moved out of engagement with the grooves 737 such that the exterior housing portion 731 is free to rotate relative to the internal support structure 733.

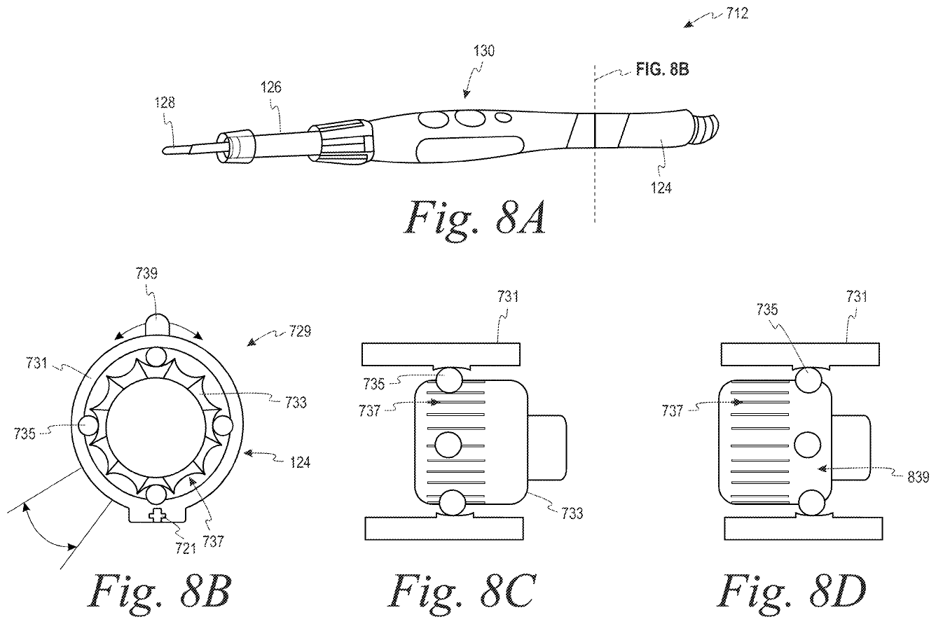

FIGS. 8A-8D depict an implementation of the electrosurgical device 712 shown in FIGS. 7A-7B and in which the rotatable mount assembly 729 includes the locking mechanism described above. FIG. 8A depicts the electrosurgical device 712, FIG. 8B depicts a cross-sectional view of the handle 124 of the electrosurgical device 712 taken through a line in shown in FIG. 8A, FIG. 8C depicts a cross-sectional view of the handle 124 of the electrosurgical device 712 taken through a line in shown in FIG. 8B and with the exterior housing portion 731 in the locked position, and FIG. 8D depicts a cross-sectional view of the handle 124 of the electrosurgical device 712 taken through a line in shown in FIG. 8B and with the exterior housing portion 731 in the unlocked position according to an example.

As shown in FIG. 8C, when the exterior housing portion 731 is in the locked position, the ball bearings 735 are received in the grooves 737 of the internal support structure 733, and an engagement between the ball bearings 735 and the grooves 737 prevents rotation of the exterior housing portion 731 relative to the internal support structure 733. As shown in FIG. 8D, when the exterior housing portion 731 is in the unlocked position, the ball bearings 735 are positioned outside of the grooves 737 and on a proximal portion 839 of the internal support structure 733 having a smaller outer diameter than the grooves 737 (e.g., a smooth surface portion). As the ball bearings 735 are disengaged from the grooves 737, the ball bearings 735 can slide along the proximal portion 839 to allow the exterior housing portion 731 to rotate relative to the internal support structure 733.

In some examples, the rotatable mount assembly 729 can include one or more features for (i) axially moving the exterior housing portion 731 between the unlocked position and the locked position, (ii) biasing the exterior housing portion 731 toward the locked position, and/or (iii) maintaining the exterior housing portion 731 at the locked position until released to move toward the unlocked position.

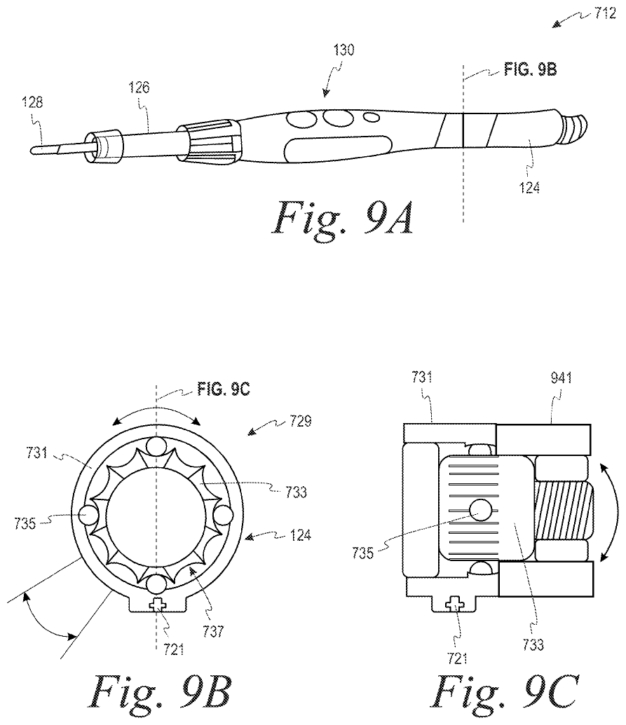

As one example, FIGS. 9A-9C depict an implementation of the electrosurgical device 712 that includes a threaded collar 941 for moving the exterior housing portion 731 between the locked position and the unlocked position. In this example, the threaded collar 941 has a first thread that couples to a second thread on the internal support structure 733. Rotating the threaded collar 941 in a first direction moves the exterior housing portion 731 toward the locked position, and rotating the threaded collar 941 in a second direction moves the exterior housing portion 931 toward the unlocked position.

In some examples, the camera 152 can be configured to move relative to at least one of the handle 124, the shaft 126, and/or the electrosurgical electrode 128. This can provide for adjusting a field of view of the camera 152 while a point of coupling between the camera assembly remains stationary relative to the handle 124 and/or the shaft 126. Within examples, the camera 152 can be adjustable in at least one dimension selected from a group consisting of: (i) roll, (ii) pitch, and (iii) yaw. Accordingly, as alternative examples, the camera 152 can be (i) adjustable in only roll, (ii) adjustable in only pitch, (iii) adjustable in only yaw, (iv) adjustable in only roll and pitch, (v) adjustable in only pitch and yaw, (vi) adjustable in only roll and yaw, or (vii) adjustable in roll, pitch, and yaw.

In some implementations, the camera 152 can be manually adjustable. In other implementations, the camera 152 can be adjustable by an electromechanical system (e.g., one or more brushless direct current (DC) motors).

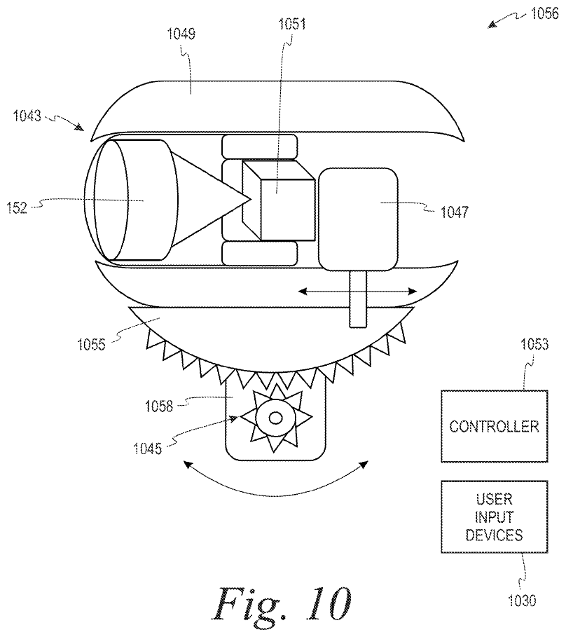

FIG. 10 depicts a camera assembly 1056 that includes a camera mount 1058, a camera 152, and an adjustment system 1043 that is configured to move the camera 152 in the at least one dimension relative to the camera mount 1058 to adjust the field of view of the camera 152. The adjustment system 1043 can include a pitch motor 1045 that is configured to adjust a pitch of an outer camera housing 1055 and the camera 152. The adjustment system 1043 can additionally or alternatively include a yaw motor 1047 that can adjust a yaw of an inner camera housing 1049 relative to the camera mount 1058 and/or the outer camera housing 1055 to thereby adjust a yaw of the camera 152. The adjustment system 1043 can additionally or alternatively include a roll motor 1051 that can rotate the camera 152 relative to the camera mount 1058, the inner camera housing 1049, and/or the outer camera housing 1055.

In some examples, the adjustment system 1043 can be configured to automatically move the camera 152 to maintain a target within the field of view of the camera 152 while the electrosurgical device 112 is being moved. This can help to more efficiently perform an electrosurgical procedure.

For instance, as shown in FIG. 10, the adjustment system 1043 can be communicatively coupled with a controller 1053 that can provide control signals to the adjustment system 1043 to operate the pitch motor 1045, the yaw motor 1047, and/or the roll motor 1051. Within examples, the controller 1053 can be implemented using hardware, software, and/or firmware. For instance, the controller 1053 can include one or more processors and a non-transitory computer readable medium (e.g., volatile and/or non-volatile memory) that stores machine language instructions or other executable instructions. The instructions, when executed by the one or more processors, cause the adjustment system 1043 to carry out the various operations described herein. The controller 1053, thus, can receive data (e.g., images captured by the camera 152) and store the data in the memory as well. The controller 1053 can be communicatively coupled with the adjustment system 1043 and/or the camera 152 via wired and/or a wireless connection.

In other examples, the adjustment system 1043 can be additionally or alternatively coupled to one or more user input device 1030 that can be operated by a user to cause the adjustment system 1043 to move the camera 152 relative to the camera mount 1058. The user input device(s) 1030 can be (i) on the handle 124 of the electrosurgical device 112 (e.g., the user input devices 130), (ii) on the electrosurgical generator 110 (e.g., the user interface 116), and/or (iii) on another device (e.g., a display device).

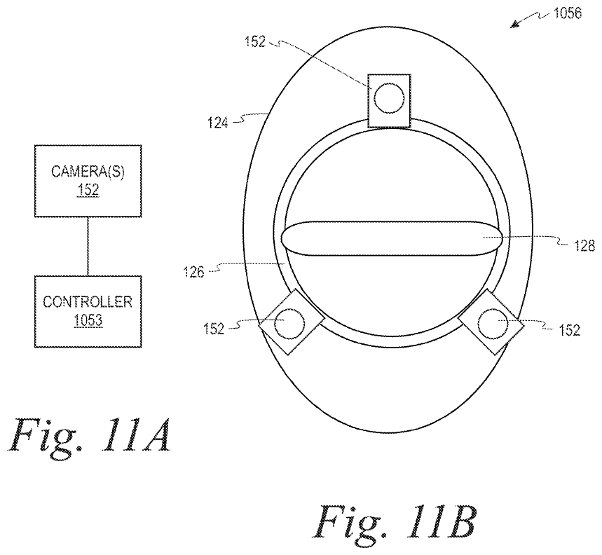

FIG. 11A is a simplified block diagram of the camera(s) 152 communicatively coupled to the controller 1053, according to an example. Within examples, the controller 1053 can also be communicatively coupled to the adjustment system 1043, or the adjustment system 1043 can be omitted from the electrosurgical device 112 that includes the camera(s) 152.

In some examples, the controller 1053 can be configured to automatically calibrate the camera(s) 152 based on an image captured by the camera(s) 152. For instance, in an implementation, the camera(s) 152 can capture an image of a reference portion of the electrosurgical device 112 that has a known reference color value at an outset of a calibration process. The portion of the electrosurgical device having the known reference color can be, for example, a portion of the handle 124, a portion of the shaft 126, and/or a portion of the electrosurgical electrode 128 that are within a field of view of the camera(s) 152.

During the calibration process, the controller 1053 can perform an image analysis on the captured image to determine a measured color value of the reference portion of the electrosurgical device 112. The controller 1053 can then compare the measured color value to the reference color value to determine a deviation between the measured color value and the reference color value. The deviation can be caused by lighting conditions and/or one or more camera settings of the camera 152.

The controller 1053 can then send, based on the deviation, a control signal to the camera(s) 152 to adjust the one or more camera settings of the camera(s) 152. As examples, the one or more camera settings can include at least one setting selected from a group consisting of: white balance, exposure, contrast and saturation, and a color profile.

In some implementations, after adjusting the one or more camera settings of the camera(s) 152, the controller 1053 can repeat the calibration process one or more times until the adjusted camera settings result in a deviation that is less than a threshold amount.

In some examples, the controller 1053 can additionally perform an image recognition analysis on the captured image to identify the reference portion of the electrosurgical device 112. This may be beneficial in implementations in which the camera(s) 152 have fields of view that are adjustable and/or the positions of the camera(s) 152 on the electrosurgical device 112 can be adjustable. In one example, the controller 1053 can identify the reference portion of the electrosurgical device 112 by (i) extracting features (e.g., by detecting edges, keypoints, and/or shapes), (ii) matching extracted features to known templates and/or trained models, (iii) localizing a structure of the reference portion (e.g., by drawing a bounding box and/or masking around a detected area), and (iv) determining the color value of the localized structure of the reference portion.

In some implementations, the reference portion of the electrosurgical device 112 can additionally or alternatively include an optical marker that can be detected in the image by the controller 1053. For example, the electrosurgical electrode 128 can include a coating that reflects and/or refracts light to provide an identifiable optical signal that can be detected in the image captured by the camera(s) 152. For instance, the coating can include metal particles, glass beads, and/or a prismatic film that reflects and/or refracts light to provide the optical signal. In further implementations, the reference portion of the electrosurgical device 112 can include a coating that provides greater reflection and/or refraction than a surgical site, and/or the reference portion of the electrosurgical device 112 can include a coating that provides less reflection and/or refraction that the surgical site to provide an optical indication of the reference portion in the captured image.

In some examples, the camera(s) 152 can include a plurality of cameras 152 at different positions around the handle 124 and/or the shaft 126 (as shown, for example, in FIG. 11B), and the controller 1053 can be configured to receive a plurality of images from the cameras 152 create a composite image that omits at least a portion of the handle 124, at least a portion of the shaft 126, and/or at least a portion of the electrosurgical electrode 128. In some implementations, the composite image can be a selectable mode of operation that can be toggled on and off. The composite image can assist a user in better visualizing the surgical site without the visual obstruction of the handle 124, the shaft 126, and/or the electrosurgical electrode 128. This may be particularly beneficial when operating in a relatively small surgical site.

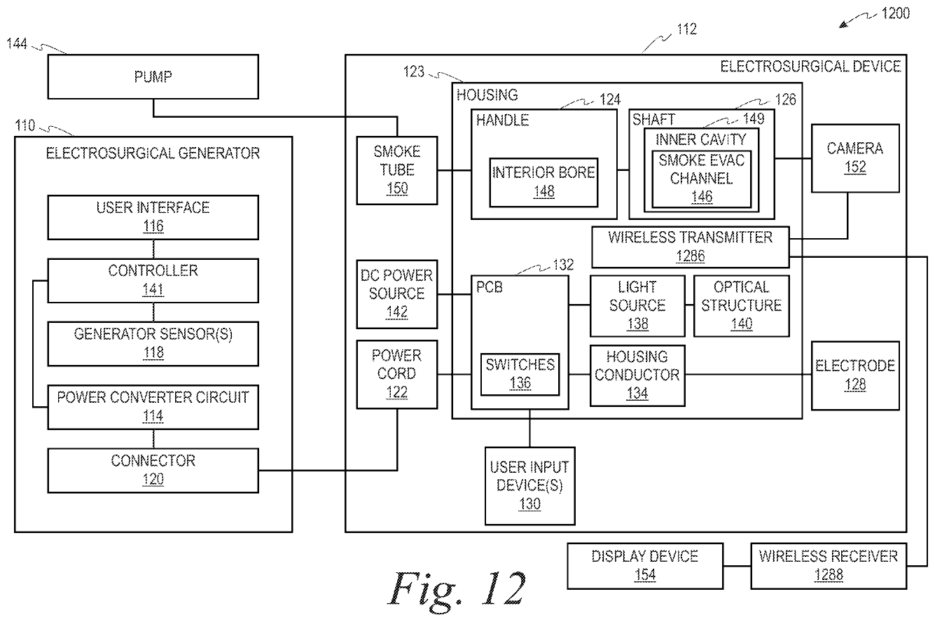

Referring now to FIG. 12, a schematic diagram of an electrosurgical system 1200 according to another example. The electrosurgical system 1200 shown in FIG. 12 is substantially similar or identical to the electrosurgical system 100 shown and described with respect to FIG. 1, except the electrosurgical system 1200 includes a wireless transmitter 1286 for transmitting the one or more images to the display device 154. Accordingly, the electrosurgical device 112 shown in FIG. 12 can be implemented as described above for the electrosurgical device 212 shown in FIGS. 2A-2D, the electrosurgical device 312 shown in FIGS. 3A-3C, the electrosurgical device 412 shown in FIG. 4B, the electrosurgical device 512 shown in FIG. 5, the electrosurgical device 612 shown in FIG. 6A, the electrosurgical device 712 shown in FIGS. 7A-9C, and/or any combination of those implementations. Additionally, as described above, the electrosurgical device 112 shown in FIG. 12 can omit one or more of the optional components described above (e.g., the smoke tube 150, the DC power source 142, the smoke evacuation channel 146, the light source 138, the optical structure 140) and/or the camera 152.

As shown in FIG. 12, the electrosurgical device 112 includes the wireless transmitter 1286 in communication with the camera 152. As described above, the camera 152 is configured to capture the one or more images. The wireless transmitter 1286 is configured to receive one or more images from the camera 152 and wirelessly communicate the one or more images to a wireless receiver 1288, which is in communication with the display device 154.

As examples, the wireless transmitter 1286 be communicatively coupled to the wireless receiver 1288 by a local area network (LAN), a wide area network (WAN), Internet, cloud, and/or near-field communications. In one example, the wireless transmitter 1286 can be configured to wirelessly communicate the one or more images to the wireless receiver 1288 using Bluetooth, and the wireless receiver 1288 can then transmit the one or more images to the display device 154 (e.g., via a wired connection). Wirelessly coupling the camera 152 to the display device 154 by the wireless transmitter 1286 and the wireless receiver 1288 can help to reduce a quantity and/or a size of cables extending within the housing 123 and/or extending externally from the electrosurgical device 112 to one or more external devices (e.g., the electrosurgical generator 110 and/or the display device 154). This can help to enhance a maneuverability of the electrosurgical device 112 and/or simplify operation of the electrosurgical device 112.

In FIG. 12, the wireless transmitter 1286 and the PCB 132 are shown as separate components. However, the wireless transmitter 1286 can include the PCB 132 and/or another printed circuit board disposed in the handle 124 and/or the shaft 126, and the printed circuit board is coupled to the camera 152 by one or more wires extending within the handle 124 and/or the shaft 126.

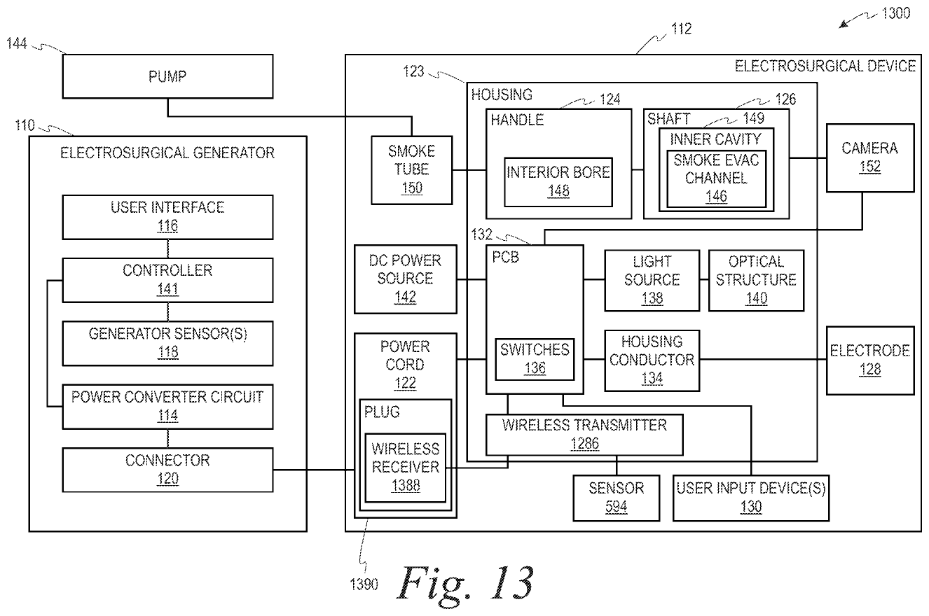

Referring now to FIG. 13, a schematic diagram of an electrosurgical system 1300 according to another example. The electrosurgical system 1300 shown in FIG. 13 is substantially similar or identical to the electrosurgical system 100 shown and described with respect to FIG. 1 and/or the electrosurgical system shown and described with respect to FIG. 12, except the electrosurgical system 1300 is configured to wirelessly transmit control signals to start and stop the supply of the electrosurgical energy from the electrosurgical generator 110 to the electrosurgical device 112. Accordingly, the electrosurgical device 112 shown in FIG. 13 can be implemented as described above for the electrosurgical device 212 shown in FIGS. 2A-2D, the electrosurgical device 312 shown in FIGS. 3A-3C, the electrosurgical device 412 shown in FIG. 4B, the electrosurgical device 512 shown in FIG. 5, the electrosurgical device 612 shown in FIG. 6A, the electrosurgical device 712 shown in FIGS. 7A-9C, and/or any combination of those implementations. Additionally, as described above, the electrosurgical device 112 shown in FIG. 13 can omit one or more of the optional components described above (e.g., the smoke tube 150, the DC power source 142, the smoke evacuation channel 146, the light source 138, the optical structure 140) and/or the camera 152.

As described above, the electrosurgical device 112 includes the one or more user input devices 130 that are operable to control operation of the electrosurgical device 112. In some examples, the one or more user input devices 130 can be on the handle 124. In another example, the one or more user input device 130 can additionally or alternatively include a foot pedal that is external to the housing 123. As shown in FIG. 13, the one or more user input devices 130 are operatively coupled to the wireless transmitter 1286 in the housing 123 (e.g., in the handle 124 and/or in the shaft 126). For instance, the one or more user input device 130 can be coupled to the wireless transmitter 1286 by the PCB 132 and/or the switches 136. More generally, the one or more user input devices 130 are coupled to the wireless transmitter 1286 such that actuation of the one or more user input devices 130 causes the wireless transmitter 1286 to wirelessly transmit a control signal indicative of the actuation of the one or more user input devices 130. As such, the wireless transmitter 1286 is configured to transmit the control signal to an electrosurgical generator 110 in response to the one or more user input devices 130 being operated.

Additionally, as described above, the power cord 122 can include a plug 1390, which can be coupled to a socket of the connector 120 of the electrosurgical generator 110. In this example, the plug 1390 can include a wireless receiver 1388 in wireless communication with the wireless transmitter 1286. In this arrangement, the wireless receiver 1388 can wirelessly receive the control signal indicative of the actuation of the one or more user input devices 130, and transmit a signal to the electrosurgical generator 110 (e.g., via a wired connection) and cause the electrosurgical generator 110 to supply or cease supplying the electrosurgical energy to the electrosurgical device via the power cord 122.

The electrosurgical device 112 that can wirelessly control signals between the housing 123 and the plug 1390 can help to reduce a quantity and size of wires in the power cord 122. For instance, some existing electrosurgical devices include at least three core wires in the power cord, including a first wire for the supply of the electrosurgical energy, a second wire for communicating a control signal indicative of actuation of a user input device for a cut mode of operation, and a third wire for communicating a control signal indicative of actuation of a user input device for a coagulation mode of operation. By contrast, the electrosurgical device 112 shown in FIG. 13 can include a single wire in the power cord 122 (e.g., for supplying the electrosurgical energy) while providing the same functionality as prior devices including three wires in the power cord 122. This can help to reduce a thickness of the power cord 122 connecting the plug 1390 to the electrosurgical device 112 to increase maneuverability of the electrosurgical device 112 throughout electrosurgical operations.

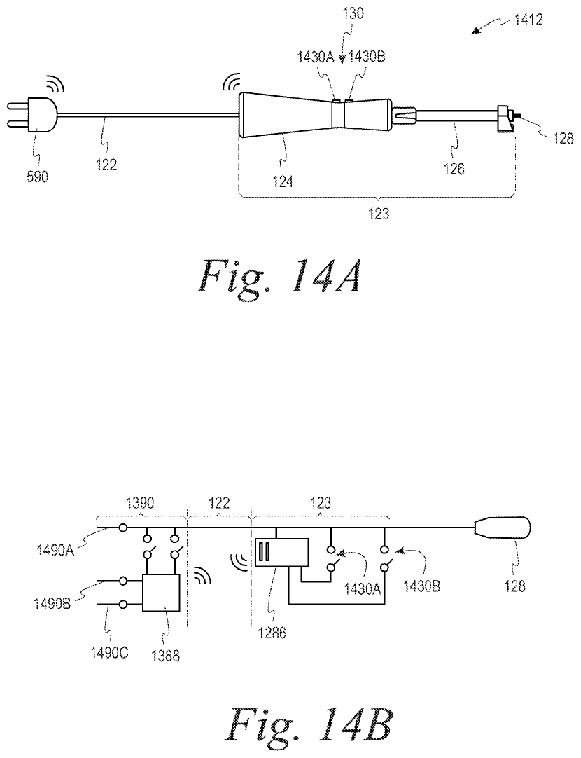

FIGS. 14A-14B depicts an electrosurgical device 1412 as an implementation of the electrosurgical device 112, according to one example. In particular, FIG. 14A depicts a side view of the electrosurgical device 1412 and FIG. 14B depicts a simplified circuit schematic diagram of the electrosurgical device 1412, according to the example. As shown in FIG. 14A, the electrosurgical device 1412 includes the housing 123, the handle 124, the shaft 126, the electrosurgical electrode 128, the power cord 122, and the plug 1390.

As shown in FIGS. 14A-14B, the electrosurgical device 1412 can include the wireless transmitter 1286 in the housing 123 (e.g., in the handle 124), and the wireless receiver 1388 in the plug 1390. The power cord 122 can include a single wire for supplying the electrosurgical energy from the electrosurgical generator 110 to the electrosurgical device 112. In this example, the power cord 122 consists of the single wire. However, in other examples, the power cord 122 can include one or more other wires for purposes other than transmitting the control signals from the electrosurgical device 112 to the electrosurgical generator 110 (e.g., one or more wires for transmitting a DC power signal from a DC power source in the electrosurgical generator 110 and/or a DC power source in the plug 1390 and/or along the power cord 122 between the plug 1390 and the housing 123).

In this example, the one or more user input devices 130 include a first user input device 1430A that is operable to cause the electrosurgical generator 110 to supply a first electrosurgical energy for a cut mode of operation, and a second user input device 1430B that is operable to cause the electrosurgical generator 110 to supply a second electrosurgical energy for a coagulation mode of operation. The first electrosurgical energy can have a first level of power and a first waveform, and the second electrosurgical energy can have a second level of power and a second waveform. The first level of power is different than the second level of power and the first waveform is different than the second waveform.

As shown in FIG. 14B, the plug 1390 can include a plurality of prongs 1490A, 1490B, 1490C that are configured to be received in respective sockets of the connector 120 of the electrosurgical generator 110. In the example shown in FIG. 14B, the prongs 1490A, 1490B, 1490C include a first prong 1490A, a second prong 1490B, and a third prong 1490C. The first prong 1490A can couple the electrosurgical generator 110 to the wire of the power cord 122 for supplying the electrosurgical energy from the electrosurgical generator 110 to the electrosurgical device 1412. The second prong 1490B and the third prong 1490C can be configured to provide the control signals to the electrosurgical generator 110, For example, the plug 1390 can be configured such that actuation of the first user input device 1430A causes the wireless transmitter 1286 to transmit a control signal to the wireless receiver 1388, which uses the second prong 1490B to signal the electrosurgical generator 110 to supply the electrosurgical energy with the first level of power and the first waveform to the power cord 122 via the first prong 1490A. Additionally, for example, the plug 1390 can be configured such that actuation of the second user input device 1430B causes the wireless transmitter 1286 to transmit a control signal to the wireless receiver 1388, which uses the third prong 1490C to signal the electrosurgical generator 110 to supply the electrosurgical energy with the second level of power and the second waveform to the power cord 122 via the first prong 1490A.

In some examples, the wireless transmitter 1286 and the wireless receiver 1388 can be paired using a hardcoded unique passkey (e.g., including a device unique serial number) and an encryption key embedded in the wireless transmitter 1286 and the wireless receiver 1388. In some examples, data communication between the wireless transmitter 1286 and the wireless receiver 1388 can also use the hardcoded unique passkey and the encryption key. For instance, after the wireless transmitter 1286 and the wireless receiver 1388 are paired using the hardcoded unique passkey and a datalink is established, then communication between the wireless transmitter 1286 and the wireless receiver 1388 can be encrypted to mitigate cross communication due to other wireless devices in proximity of the electrosurgical device 1412.

In some implementations, one or more components within the housing 123 and the plug 1390 can be battery powered, and responsive to the plug 1390 being coupled to the connector 120 of the electrosurgical generator 110 and powered up, the wireless receiver 1388 of the plug 1390 can establish the datalink via unique passkey and start data communication with the wireless transmitter 1286 in the housing 123. As such, the wireless receiver 1388 and the wireless transmitter 1286 can have a master and slave relationship, respectively, in some examples.

In one example operation, after the wireless receiver 1388 and the wireless transmitter 1286 are paired successfully, the wireless receiver 1388 can also connect to an available Wi-Fi network by using name (SSID) and Wi-Fi network password (WPA2 key) and establish a secure link with cloud database to transmit operational data based on a sensor 594 (e.g., a cutting operation duty cycle, a coagulation duty cycle, a temperature of the electrosurgical electrode 128, a temperature of the shaft 126, a temperature of the handle 124, a battery level, a strength of suction for smoke evacuation, a content of smoke evacuated, a quantity of particulates in smoke evacuated, and/or a concentration of particulates in smoke evacuated). The wireless transmitter 1286 can transmit the operational data periodically (e.g., every 5 seconds, every 10 seconds, etc.), or continuously. In some examples, the operational data can be displayed to the user while the user is operating the electrosurgical device 1412 (e.g., via the display device 154 shown in FIGS. 1 and 12).

The user can control whether the “cut” or “coag” function is activated via the user input devices 130 on the electrosurgical device 1412. Whichever function is selected, wireless transmitter 1286 can send an encrypted corresponding code to the wireless receiver 1388 of the plug 1390. The wireless receiver 1388 can decode the received data pack and active the either Cut operation or Coagulation operation whichever was selected using the user input devices 130, and after activation the wireless receiver 1388 can return the acknowledgment back to wireless transmitter 1286.

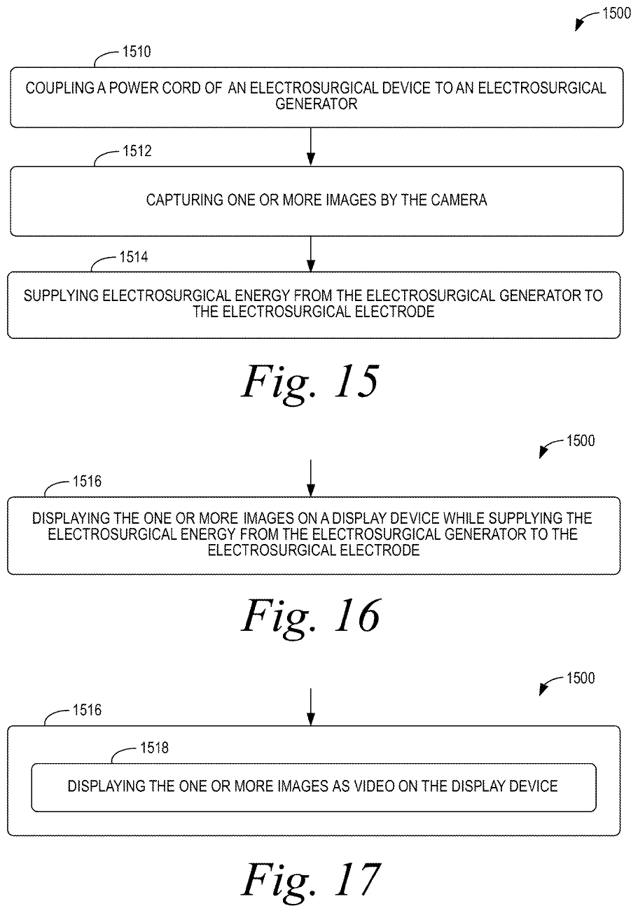

Referring now to FIG. 15, a flowchart for a process 1500 of operating an electrosurgical device is shown according to an example. At block 1510, the process 1500 includes coupling a power cord of an electrosurgical device to an electrosurgical generator. The electrosurgical device includes (a) a handle extending between a proximal handle end and a distal handle end, (b) a shaft extending from the distal handle end of the handle, where the shaft extends between a proximal shaft end and a distal shaft end, (c) an electrosurgical electrode extending from the distal shaft end, and (d) a camera assembly coupled to the distal shaft end. The camera assembly includes: (i) a first housing portion including a through-bore receiving the distal shaft end, (ii) a second housing portion extending laterally outward from the first housing portion and the shaft, where the second housing portion comprises a camera mount surface extending in a plane that has a normal line extending toward the electrosurgical electrode, and (iii) a camera coupled to the camera mount surface.

After coupling the electrosurgical device to the electrosurgical generator at block 1510, the process 1500 includes capturing one or more images by the camera at block 1512. While capturing the one or more images by the camera at block 1512, the process 1500 includes supplying electrosurgical energy from the electrosurgical generator to the electrosurgical electrode at block 1514.

FIGS. 16-24 depict additional aspects of the process 1500 according to further examples. As shown in FIG. 16, the process 1500 can also include displaying the one or more images on a display device at block 1516 while supplying the electrosurgical energy from the electrosurgical generator to the electrosurgical electrode at block 1514.

As shown in FIG. 17, displaying the one or more images on a display device at block 1516 can include displaying the one or more images as video on the display device at block 1518.

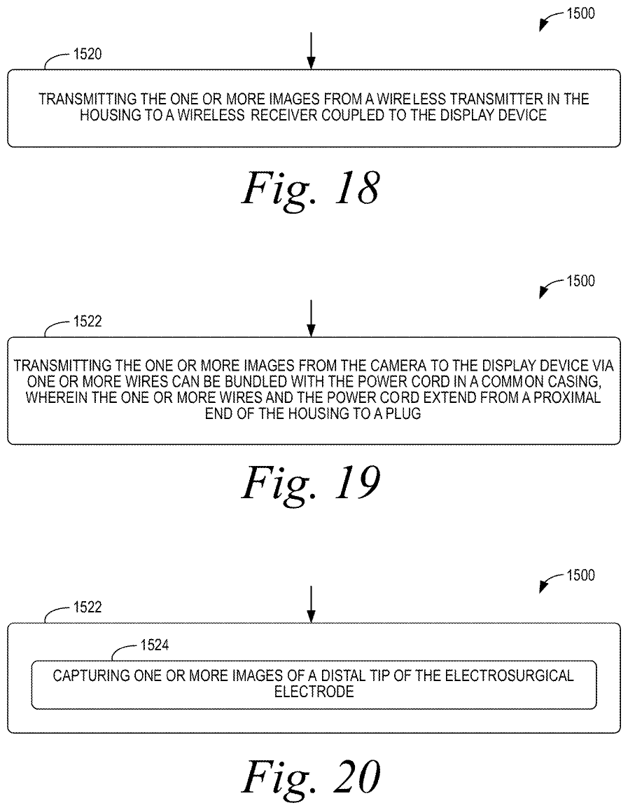

As shown in FIG. 18, the process 1500 can further include transmitting the one or more images from a wireless transmitter in the housing to a wireless receiver coupled to the display device at block 1520.

As shown in FIG. 19, the process 1500 can further include transmitting the one or more images from the camera to the display device via one or more wires can be bundled with the power cord in a common casing, wherein the one or more wires and the power cord extend from a proximal end of the housing to a plug at block 1522.

As shown in FIG. 20, capturing the one or more images at block 1512 can include capturing one or more images of a distal tip of the electrosurgical electrode at block 1524.

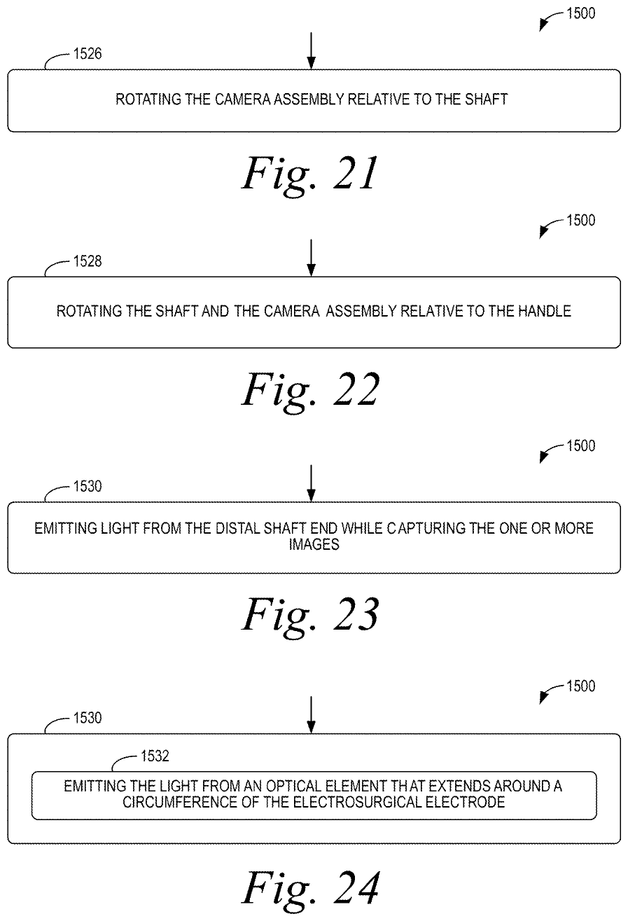

As shown in FIG. 21, the process 1500 can include rotating the camera assembly relative to the shaft at block 1526.

As shown in FIG. 22, the process 1500 can include rotating the shaft and the camera assembly relative to the handle at block 1528.

As shown in FIG. 23, the process 1500 can include emitting light from the distal shaft end at block 1530 while capturing the one or more images at block 1512.

As shown in FIG. 24, emitting the light from the distal shaft end at block 1530 can include emitting the light from an optical element that extends around a circumference of the electrosurgical electrode at block 1532.

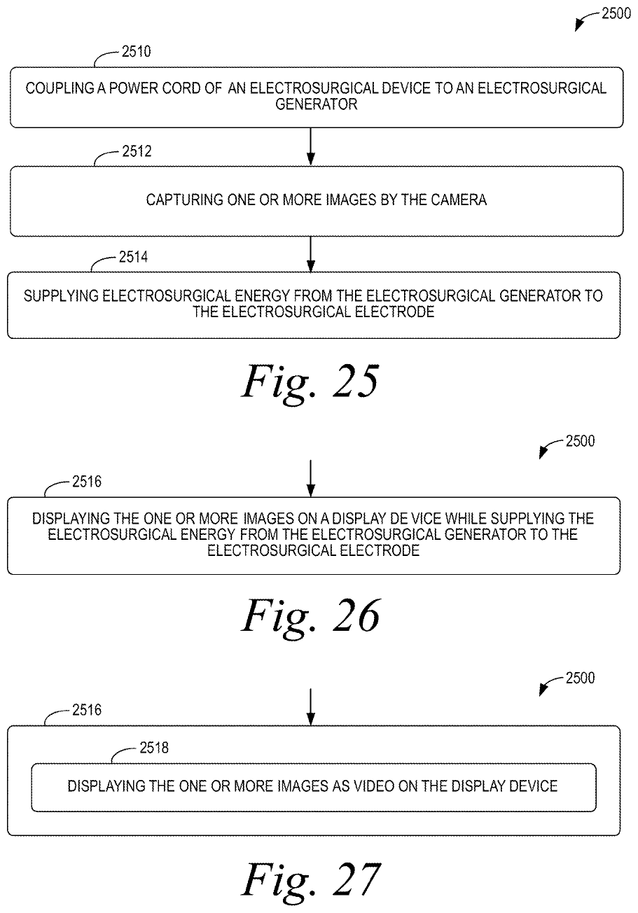

Referring now to FIG. 25, a flowchart for a process 2500 of operating an electrosurgical device is shown according to an example. At block 2510, the process 2500 includes coupling a power cord of an electrosurgical device to an electrosurgical generator. The electrosurgical device includes (i) a handle extending between a proximal handle end and a distal handle end, (ii) a shaft extending from the distal handle end of the handle, where the shaft extends between a proximal shaft end and a distal shaft end, (iii) a camera at the distal shaft end, and (iv) an electrosurgical electrode coupled to an exterior surface the shaft and extending distally of the distal shaft end. A longitudinal axis of the electrosurgical electrode is parallel to a longitudinal axis of the shaft.

After coupling the electrosurgical device to the electrosurgical generator at block 2510, the process 2500 includes capturing one or more images by the camera at block 2512. While capturing the one or more images by the camera at block 2512, the process 2500 includes supplying electrosurgical energy from the electrosurgical generator to the electrosurgical electrode at block 2514.

FIGS. 26-34 depict additional aspects of the process 2500 according to further examples. As shown in FIG. 26, the process 2500 can also include displaying the one or more images on a display device at block 2516 while supplying the electrosurgical energy from the electrosurgical generator to the electrosurgical electrode at block 2514.

As shown in FIG. 27, displaying the one or more images on a display device at block 2516 can include displaying the one or more images as video on the display device at block 2518.

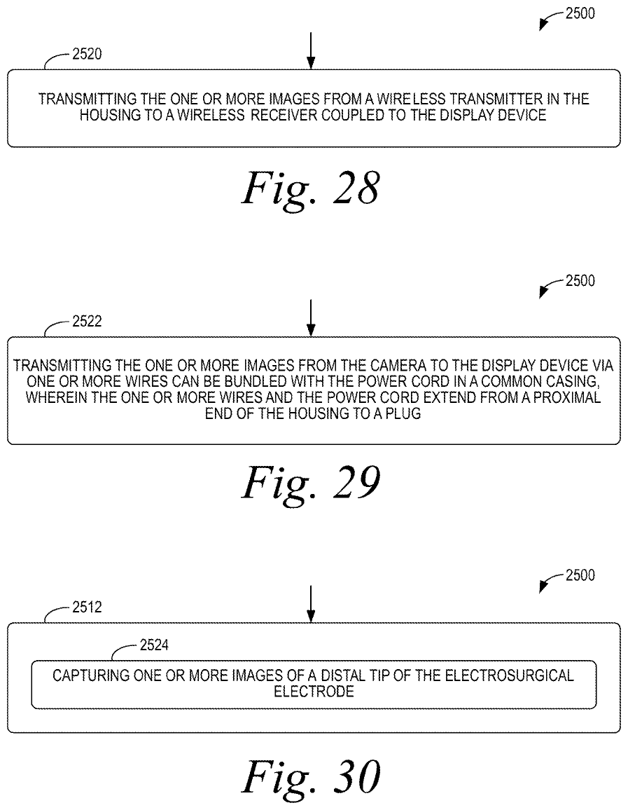

As shown in FIG. 28, the process 2500 can further include transmitting the one or more images from a wireless transmitter in the housing to a wireless receiver coupled to the display device at block 2520.

As shown in FIG. 29, the process 2500 can further include transmitting the one or more images from the camera to the display device via one or more wires can be bundled with the power cord in a common casing, wherein the one or more wires and the power cord extend from a proximal end of the housing to a plug at block 2522.

As shown in FIG. 30, capturing the one or more images at block 2512 can include capturing one or more images of a distal tip of the electrosurgical electrode at block 2524.

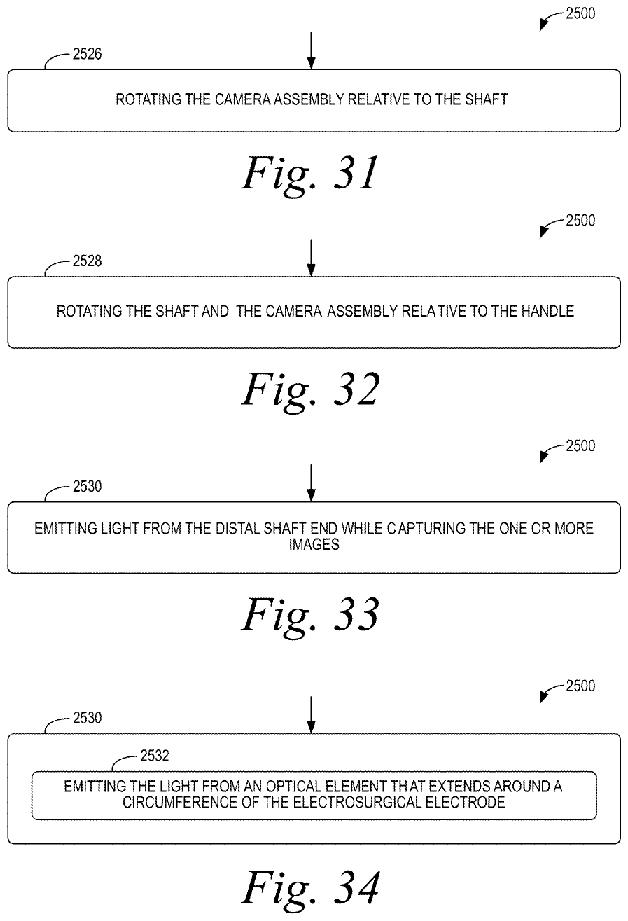

As shown in FIG. 31, the process 2500 can include rotating the camera assembly relative to the shaft at block 2526.

As shown in FIG. 32, the process 2500 can include rotating the shaft and the camera assembly relative to the handle at block 2528.

As shown in FIG. 33, the process 2500 can include emitting light from the distal shaft end at block 2530 while capturing the one or more images at block 2512.

As shown in FIG. 34, emitting the light from the distal shaft end at block 2530 can include emitting the light from an optical element that extends around a circumference of the electrosurgical electrode at block 2532.

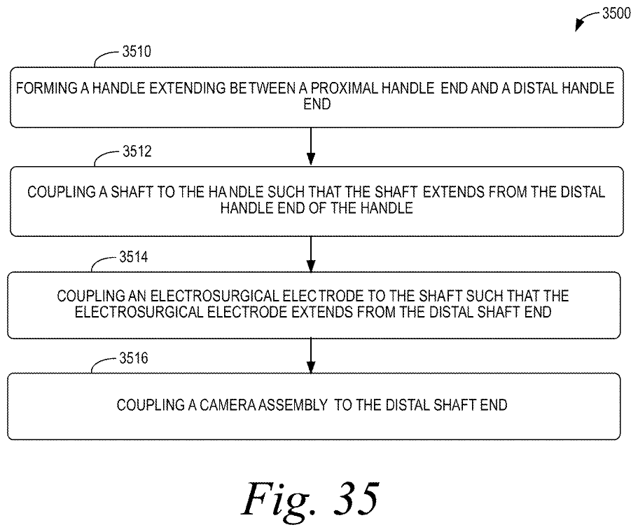

Referring now to FIG. 35, a flowchart for a process 3500 of forming an electrosurgical device is shown according to an example. At block 3510, the process 3500 includes forming a handle extending between a proximal handle end and a distal handle end. At block 3512, the process includes coupling a shaft to the handle such that the shaft extends from the distal handle end of the handle. The shaft extends between a proximal shaft end and a distal shaft end. At block 3514, the process 3500 includes coupling an electrosurgical electrode to the shaft such that the electrosurgical electrode extends from the distal shaft end. At block 3516, the process 3500 includes coupling a camera assembly to the distal shaft end. The camera assembly includes a first housing portion including a through-bore receiving the distal shaft end. The camera assembly also includes a second housing portion extending laterally outward from the first housing portion and the shaft. The second housing portion includes a camera mount surface extending in a plane that has a normal line extending toward the electrosurgical electrode. The camera assembly further includes a camera coupled to the camera mount surface.

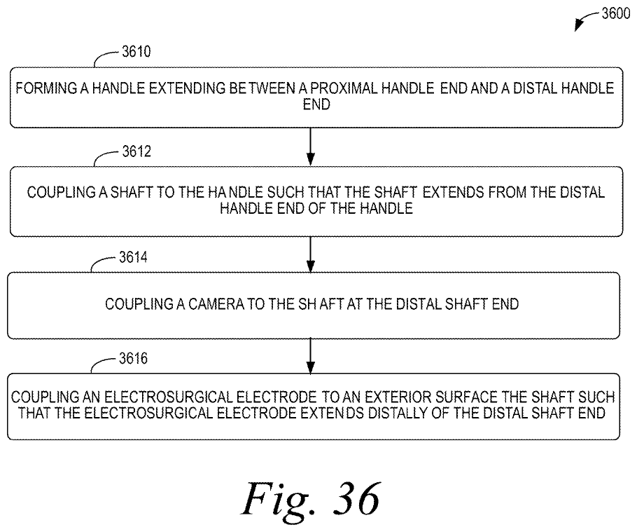

Referring now to FIG. 36, a flowchart for a process 3600 of forming an electrosurgical device is shown according to an example. At block 3610, the process 3600 includes forming a handle extending between a proximal handle end and a distal handle end. At block 3612, the process 3600 includes coupling a shaft to the handle such that the shaft extends from the distal handle end of the handle. The shaft extends between a proximal shaft end and a distal shaft end. At block 3614, the process 3600 includes coupling a camera to the shaft at the distal shaft end. At block 3616, the process 3600 includes coupling an electrosurgical electrode to an exterior surface the shaft such that the electrosurgical electrode extends distally of the distal shaft end. A longitudinal axis of the electrosurgical electrode is parallel to a longitudinal axis of the shaft.

The description of the different advantageous arrangements has been presented for purposes of illustration and description, and is not intended to be exhaustive or limited to the embodiments in the form disclosed. Many modifications and variations will be apparent to those of ordinary skill in the art. Further, different advantageous embodiments may provide different advantages as compared to other advantageous embodiments. The implementation or implementations selected are chosen and described in order to best explain the principles of the embodiments, the practical application, and to enable others of ordinary skill in the art to understand the disclosure for various embodiments with various modifications as are suited to the particular use contemplated.

What is claimed is:

1. An electrosurgical device, comprising:
a handle extending between a proximal handle end and a distal handle end;
a shaft extending from the distal handle end of the handle, wherein the shaft extends between a proximal shaft end and a distal shaft end;
an electrosurgical electrode extending from the distal shaft end; and
a camera assembly coupled to the distal shaft end, wherein the camera assembly comprises:
(i) a first housing portion including a through-bore receiving the distal shaft end,
(ii) a second housing portion extending laterally outward from the first housing portion and the shaft, wherein the second housing portion comprises a camera mount surface extending in a plane that has a normal line extending toward the electrosurgical electrode, and
(iii) a camera coupled to the camera mount surface.

2. The electrosurgical device of claim 1, wherein the first housing portion further comprises an aperture at a distal end of the camera assembly, wherein the electrosurgical electrode extends distally through the aperture at the distal end of the camera assembly.

3. The electrosurgical device of claim 2, further comprising a light source configured to emit light through the aperture at the distal end of the camera assembly.

4. The electrosurgical device of claim 2, wherein the aperture at the distal end of the camera assembly is coaxial with a longitudinal axis of the electrosurgical electrode and a longitudinal axis of the shaft.

5. The electrosurgical device of claim 1, wherein the camera is coupled to the camera mount surface such that a field of view of the camera includes a distal tip of the electrosurgical electrode.

6. The electrosurgical device of claim 1, wherein the first housing portion is configured to rotate about the shaft while the distal shaft end is received in the through-bore.

7. The electrosurgical device of claim 1, wherein the first housing portion is rotationally fixed relative to the shaft.

8. The electrosurgical device of claim 1, further comprising a wireless transmitter in communication with the camera, wherein the wireless transmitter is configured to receive one or more images from the camera and wirelessly communicate the one or more images to a wireless receiver, which is in communication with a display device

9. The electrosurgical device of claim 8, wherein the wireless transmitter is configured to wirelessly communicate the one or more images to the wireless receiver using Bluetooth.

10. The electrosurgical device of claim 8, wherein the wireless transmitter comprises a printed circuit board disposed in the handle, wherein the printed circuit board is coupled to the camera by one or more wires extending within the handle and the shaft.

11. The electrosurgical device of claim 8, further comprising one or more user input devices on the handle, wherein the one or more user input devices are operable to control operation of the electrosurgical device.

12. The electrosurgical device of claim 11, wherein the one or more user input devices are operatively coupled to the wireless transmitter in the handle, wherein the wireless transmitter is configured to transmit a control signal to an electrosurgical generator in response to the one or more user input devices being operated.

13. The electrosurgical device of claim 12, wherein the one or more user input devices comprise:
a first user input device that is operable to cause the electrosurgical generator to supply a first electrosurgical energy for a cut mode of operation; and
a second user input device that is operable to cause the electrosurgical generator to supply a second electrosurgical energy for a coagulation mode of operation,
wherein the first electrosurgical energy has a first level of power and a first waveform, and the second electrosurgical energy has a second level of power and a second waveform, and
wherein the first level of power is different than the second level of power and the first waveform is different than the second waveform.

14. The electrosurgical device of claim 12, further comprising a power cord that is configured to receive electrosurgical energy from the electrosurgical generator, wherein the power cord consists of a single wire within an insulator.

15. An electrosurgical device, comprising:
a handle extending between a proximal handle end and a distal handle end;
a shaft extending from the distal handle end of the handle, wherein the shaft extends between a proximal shaft end and a distal shaft end;
a camera at the distal shaft end; and
an electrosurgical electrode coupled to an exterior surface the shaft and extending distally of the distal shaft end,
wherein a longitudinal axis of the electrosurgical electrode is parallel to a longitudinal axis of the shaft.

16. The electrosurgical device of claim 15, wherein the electrosurgical electrode is coupled to the exterior surface of the shaft by a collar, wherein the collar comprises:
a first collar portion that extends around the exterior surface of the shaft at a distal portion of the shaft; and
a second collar portion that couples the electrosurgical electrode to the first collar portion.

17. The electrosurgical device of claim 16, wherein the second collar portion defines an elongated slot for receiving a proximal portion of the electrosurgical electrode.

18. The electrosurgical device of claim 16, wherein the electrosurgical electrode is electrically coupled to the shaft such that electrosurgical energy is supplied from an electrosurgical generator to the electrosurgical electrode via the shaft.

19. The electrosurgical device of claim 18, wherein the collar comprises a conductor that extends from the through-bore of the first collar portion to the elongated slot of the second collar portion to electrically couple the electrosurgical electrode to the shaft.

20. The electrosurgical device of claim 15, further comprising a light source and an optical element that are configured to emit light from an aperture at the distal shaft end.

21. The electrosurgical device of claim 20, wherein the optical element extends around a circumference of the camera.

22. The electrosurgical device of claim 16, wherein the first collar portion is configured to rotate about the shaft while the distal shaft end.

23. The electrosurgical device of claim 16, wherein the first collar portion is rotationally fixed relative to the shaft.

24. The electrosurgical device of claim 15, further comprising a wireless transmitter in communication with the camera, wherein the wireless transmitter is configured to receive one or more images from the camera and wirelessly communicate the one or more images to a wireless receiver, which is in communication with a display device.

25. The electrosurgical device of claim 24, wherein the wireless transmitter is configured to wirelessly communicate the one or more images to the wireless receiver using Bluetooth.

26. The electrosurgical device of claim 24, wherein the wireless transmitter comprises a printed circuit board disposed in the handle, wherein the printed circuit board is coupled to the camera by one or more wires extending within the handle and the shaft.

27. The electrosurgical device of claim 24, further comprising one or more user input devices on the handle, wherein the one or more user input devices are operable to control operation of the electrosurgical device.

28. The electrosurgical device of claim 27, wherein the one or more user input devices are operatively coupled to the wireless transmitter in the handle, wherein the wireless transmitter is configured to transmit a control signal to an electrosurgical generator in response to the one or more user input devices being operated.

29. The electrosurgical device of claim 28, wherein the one or more user input devices comprise:
a first user input device that is operable to cause the electrosurgical generator to supply a first electrosurgical energy for a cut mode of operation; and
a second user input device that is operable to cause the electrosurgical generator to supply a second electrosurgical energy for a coagulation mode of operation,
wherein the first electrosurgical energy has a first level of power and a first waveform, and the second electrosurgical energy has a second level of power and a second waveform, and
wherein the first level of power is different than the second level of power and the first waveform is different than the second waveform.

30. The electrosurgical device of claim 28, further comprising a power cord that is configured to receive electrosurgical energy from the electrosurgical generator, wherein the power cord consists of a single wire within an insulator.

31. A method of operating an electrosurgical device, comprising:
coupling a power cord of an electrosurgical device to an electrosurgical generator, wherein the electrosurgical device comprises:
a handle extending between a proximal handle end and a distal handle end;
a shaft extending from the distal handle end of the handle, wherein the shaft extends between a proximal shaft end and a distal shaft end;
an electrosurgical electrode extending from the distal shaft end; and
a camera assembly coupled to the distal shaft end, wherein the camera assembly comprises:
(i) a first housing portion including a through-bore receiving the distal shaft end,
(ii) a second housing portion extending laterally outward from the first housing portion and the shaft, wherein the second housing portion comprises a camera mount surface extending in a plane that has a normal line extending toward the electrosurgical electrode; and
(iii) a camera coupled to the camera mount surface;
after coupling the electrosurgical device to the electrosurgical generator, capturing one or more images by the camera; and
while capturing the one or more images by the camera, supplying electrosurgical energy from the electrosurgical generator to the electrosurgical electrode.

32. The method of claim 31, further comprising displaying the one or more images on a display device while supplying the electrosurgical energy from the electrosurgical generator to the electrosurgical electrode.

33. The method of claim 32, wherein displaying the one or more images on a display device comprises displaying the one or more images as video on the display device.

34. The method of any one of claims 32-33, further comprising transmitting the one or more images from a wireless transmitter in the housing to a wireless receiver coupled to the display device.

35. The method of any one of claims 32-33, further comprising transmitting the one or more images from the camera to the display device via one or more wires can be bundled with the power cord in a common casing, wherein the one or more wires and the power cord extend from a proximal end of the housing to a plug.

36. The method of any one of claims 31-35, wherein capturing the one or more images comprises capturing one or more images of a distal tip of the electrosurgical electrode.

37. The method of any one of claims 31-36, further comprising rotating the camera assembly relative to the shaft.

38. The method of any one of claims 31-37, further comprising rotating the shaft and the camera assembly relative to the handle.

39. The method of any one of claims 31-38, further comprising emitting light from the distal shaft end while capturing the one or more images.

40. The method of claim 39, wherein emitting the light from the distal shaft end comprises emitting the light from an optical element that extends around a circumference of the electrosurgical electrode.

41. A method of operating an electrosurgical device, comprising:
coupling a power cord of an electrosurgical device to an electrosurgical generator, wherein the electrosurgical device comprises:
a handle extending between a proximal handle end and a distal handle end,
a shaft extending from the distal handle end of the handle, wherein the shaft extends between a proximal shaft end and a distal shaft end,
a camera at the distal shaft end,
an electrosurgical electrode coupled to an exterior surface the shaft and extending distally of the distal shaft end,
wherein a longitudinal axis of the electrosurgical electrode is parallel to a longitudinal axis of the shaft;
after coupling the electrosurgical device to the electrosurgical generator, capturing one or more images by the camera; and
while capturing the one or more images by the camera, supplying electrosurgical energy from the electrosurgical generator to the electrosurgical electrode.

42. The method of claim 41, further comprising displaying the one or more images on a display device while supplying the electrosurgical energy from the electrosurgical generator to the electrosurgical electrode.

43. The method of claim 42, wherein displaying the one or more images on a display device comprises displaying the one or more images as video on the display device. The method of any one of claims 42-43, further comprising transmitting the one or more images from a wireless transmitter in the housing to a wireless receiver coupled to the display device.

45. The method of any one of claims 42-43, further comprising transmitting the one or more images from the camera to the display device via one or more wires can be bundled with the power cord in a common casing, wherein the one or more wires and the power cord extend from a proximal end of the housing to a plug.

46. The method of any one of claims 41-45, wherein capturing the one or more images comprises capturing one or more images of a distal tip of the electrosurgical electrode.

47. The method of any one of claims 41-46, further comprising rotating the electrosurgical electrode relative to the shaft.

48. The method of any one of claims 41-47, further comprising rotating the shaft and the electrosurgical electrode relative to the handle.

49. The method of any one of claims 41-48, further comprising emitting light from the distal shaft end while capturing the one or more images.

50. The method of claim 49, wherein emitting the light from the distal shaft end comprises emitting the light from an optical element that extends around a circumference of the camera.

51. A method of forming an electrosurgical device, comprising:
forming a handle extending between a proximal handle end and a distal handle end;
coupling a shaft to the handle such that the shaft extends from the distal handle end of the handle, wherein the shaft extends between a proximal shaft end and a distal shaft end;
coupling an electrosurgical electrode to the shaft such that the electrosurgical electrode extends from the distal shaft end; and
coupling a camera assembly to the distal shaft end, wherein the camera assembly comprises:
a first housing portion including a through-bore receiving the distal shaft end, a second housing portion extending laterally outward from the first housing portion and the shaft, wherein the second housing portion comprises a camera mount surface extending in a plane that has a normal line extending toward the electrosurgical electrode; and
a camera coupled to the camera mount surface.

52. A method of forming an electrosurgical device, comprising:
forming a handle extending between a proximal handle end and a distal handle end;
coupling a shaft to the handle such that the shaft extends from the distal handle end of the handle, wherein the shaft extends between a proximal shaft end and a distal shaft end;
coupling a camera to the shaft at the distal shaft end; and
coupling an electrosurgical electrode to an exterior surface the shaft such that the electrosurgical electrode extends distally of the distal shaft end,
wherein a longitudinal axis of the electrosurgical electrode is parallel to a longitudinal axis of the shaft.
